(19) United States
(12) Patent Application Publication
(10) Pub. No.: US 2019/0382747 A1
(43) Pub. Date: Dec. 19, 2019

(54) Stabilized Proteins and Method of Making the Same

(71) Applicants: Colorado State University Research Foundation, Fort Collins, CO (US); Oregon State University, Corvallis, OR (US)

(72) Inventors: Pui Shing Ho, Fort Collins, CO (US); Anna-Carin C. Carlsson, Kungsbacka (SE); Matthew R. Scholfield, Fort Collins, CO (US); Melissa C. Ford, Sellersville, PA (US); Rhianon Kay Rowe Hartje, Fort Collins, CO (US); Austin T. Alexander, Corvallis, OR (US); Ryan A. Mehl, Corvallis, OR (US)

(73) Assignees: Colorado State University Research Foundation, Fort Collins, CO (US); Oregon State University, Corvallis, OR (US)

(21) Appl. No.: 16/444,566

(22) Filed: Jun. 18, 2019

Related U.S. Application Data
(60) Provisional application No. 62/686,339, filed on Jun. 18, 2018.

(51) Int. Cl. C12N 9/96; C07K 1/107; C07K 14/47; C12N 9/36

(52) U.S. Cl. CPC C12N 9/96; C07K 1/1077; C12Y 302/01017; C12N 9/2462; C07K 14/4702

(57) ABSTRACT

The present disclosure relates to compositions and methods for increasing the stability of an engineered protein by halogenating at least one amino acid residue of the protein to form a stabilizing hydrogen bond-enhanced halogen bond (HeX-B).

### CROSS REFERENCE TO RELATED APPLICATIONS

This application claims the benefit of U.S. Provisional Application No. 62/686,339, filed Jun. 18, 2018, the disclosure of which is hereby incorporated by reference in its entirety.

### GOVERNMENTAL RIGHTS

This invention was made with government support under grant CHE1608146 awarded by National Science Foundation, and grant R01 GM114653 awarded by National Institutes of Health. The government has certain rights in the invention.

### FIELD OF THE INVENTION

The present disclosure relates to compositions and methods for increasing the stability of an engineered protein by halogenating at least one amino acid residue of the protein to form a stabilizing hydrogen bond-enhanced halogen bond (HeX-B or HBeXB), halogen-hydrogen bond donor interactions, any similarly named interaction involving cooperative or synergistic effects of a hydrogen bond to a halogen to enhance or increase a halogen bond, or any other improved ability of the halogen to form a halogen bond.

### BACKGROUND OF THE INVENTION

The construction of stable recombinant proteins is important in biomolecular engineering, particularly in the design of biologics-based therapeutics. Efforts to increase or enhance stability of recombinant proteins are limited by the molecular tools provided by nature. Although some approaches to stabilize recombinant proteins have been somewhat successful, rarely do these methods stabilize a protein by significantly more than 1 kcal/mol. Incorporation of non-canonical building blocks into recombinant proteins may overcome such limitations; however, such methods are constrained by the standard menu of non-covalent interactions that dictate molecular folding. As such, there is a need in the art for new non-canonical tools for molecular design. Such non-canonical tools can provide powerful means for molecular design in biomolecular engineering, medicinal chemistry, and material science that can have applications

Described herein is a non-canonical tool for stabilizing recombinant proteins, a hydrogen bond-enhanced halogen bond (HeX-B), which is a powerful tool for molecular design in biomolecular engineering, medicinal chemistry, material science, and design of biologics-based therapeutics.

### SUMMARY OF THE INVENTION

In an aspect, the disclosure provides a method of forming a hydrogen bond-enhanced halogen bond (HeX-B) by halogenating at least one amino acid residue of the protein wherein the stability of the engineered protein is higher than a parent protein under the same conditions. The halogen atom can be selected from fluorine, chlorine, bromine, or iodine. The halogen atom can be added to the at least one amino acid residue at the meta-position.

The formation of a HeX-B can comprise a halogen bond (XB) that forms an electropositive σ-hole. The formation of a HeX-B can comprise a XB that further forms an electronegative annulus around the center of the bond.

The formation of a HeX-B can comprise a hydrogen bond (HB) acting as an electron donor. The formation of a HeX-B can comprise a HB that intensifies the electropositive σ-hole.

The formation of a HeX-B can comprise an engineered protein that can be more thermally stable than the parent protein under the same conditions. The engineered protein can have a melting temperature that is at least 0.5° C. higher than the parent protein. The engineered protein can have a melting temperature that is at least 1° C. higher than the parent protein. The engineered protein can have an enthalpy (ΔH_{M}) that is more than 1 kcal/mol higher than the parent protein. The engineered protein can have an enthalpy (ΔH_{M}) that is at least 2 kcal/mol higher than the parent protein. The engineered protein can have an enthalpy (ΔH_{M}) that is at least 3 kcal/mol higher than the parent protein. The engineered protein can be an engineered enzyme. The enzymatic activity of the engineered enzyme can be higher than a parent enzyme under the same conditions.

The formation of a HeX-B can comprise at least one amino acid residue of the protein that is halogenated, wherein the halogen may be partially exposed to solvent. The formation of a HeX-B can comprise at least one amino acid residue of the protein that is halogenated, wherein the halogen may not be exposed to solvent.

In another aspect, the disclosure provides a composition comprising an engineered protein comprising a halogenated amino acid residue, wherein the halogenated amino acid residue comprises formation of a hydrogen bond-enhanced halogen bond (HeX-B) which stabilizes the engineered protein. The halogenated amino acid residue can comprise a halogen atom selected from fluorine, chlorine, bromine, or iodine. The halogen atom can be added to the amino acid residue at the meta-position.

The engineered protein can comprise a XB that forms an electropositive σ-hole. The engineered protein can comprise a XB that further forms an electronegative annulus around the center of the bond.

The engineered protein can comprise a HB acting as an electron donor. The engineered protein can comprise a HB that intensifies the electropositive σ-hole.

The thermal stability of the engineered protein can be higher than a parent protein under the same conditions.

The engineered protein can be an enzyme, a structural protein, a signaling protein, a regulatory protein, a transport protein, a sensory protein, a motor protein, a defense protein, a hormonal protein, or a storage protein.

The engineered protein can be an engineered enzyme. The enzymatic activity of the engineered enzyme can be higher than a parent enzyme under the same conditions.

The engineered protein can comprise a halogenated amino acid residue partially, wherein the halogen may be partially exposed to solvent. The engineered protein can comprise a halogenated amino acid residue, wherein the halogen may not be exposed to solvent.

The engineered protein can have a melting temperature that is at least 0.5° C. higher than the parent protein. The engineered protein can have a melting temperature that is at least 1° C. higher than the parent protein. The engineered protein can have an enthalpy (ΔH_{M}) that is at least 1 kcal/mol higher than the parent protein. The engineered protein can have an enthalpy (ΔH_{M}) that is at least 2 kcal/mol higher than the parent protein. The engineered protein can have an enthalpy (ΔH_{M}) that is at least 3 kcal/mol higher than the parent protein.

### BRIEF DESCRIPTION OF THE FIGURES

The application file contains at least one drawing executed in color. Copies of this patent application publication with color drawing(s) will be provided by the Office upon request and payment of the necessary fee.

FIG. 1 depicts an image of the amphoteric property of halogen substituents, where the anisotropic charge distribution, as predicted by the σ-hole model, allows the halogen to accept an HB from a hydroxyl and donate an XB to a carbonyl oxygen.

FIG. 2 depicts an image of the overall crystal structure of WT*-T4L (with two Cys residues replaced by Thr and Ala), which undergoes two-state reversible melting.

FIGS. 3A-3C depict an images of omit electron density maps where the Fo−Fc difference maps (at 1.2a level) were calculated after simulating annealing with the side chain at position 18 deleted for ^{mCl}Y18-T4L (FIG. 3A), ^{mBr}Y18-T4L (FIG. 3B), and ^{mI}Y18-T4L (FIG. 3C).

FIG. 4A depicts an image of the overall crystal structure of chlorinated ^{mCl}Y18-T4L (magenta backbone trace and carbon atoms) overlaid on WT*-T4L (blue trace and carbon atoms).

FIG. 4B depicts an image of the overall crystal structure of brominated ^{mBr}Y18-T4L (cyan) overlaid on WT*-T4L (blue).

FIG. 4C depicts an image of the overall crystal structure of iodinated ^{mI}Y18-T4L (orange) overlaid on WT*-T4L (blue).

FIG. 5A depicts a stereoimage of the interacting water molecules that bridge from Y18 to E11, G28, and R14 (labeled W1-W6) in WT*-T4L.

FIG. 5B depicts a stereoimage of the interacting water molecules (labeled W1-W6) in ^{mCl}Y18-T4L where the rotamer with the chlorine (emerald green) sitting inside [^{mCl}Y18-T4L(i), carbon atoms colored yellow] the loop. Waters are labeled W1-W6, with those that are in positions nearly identical to those of WT* colored and labeled in blue and those in positions unique to the i-rotamer colored and labeled in yellow (along with the carbons of the Y18 side chain).

FIG. 5C depicts a stereoimage of the interacting water molecules (labeled W1-W6) in ^{mCl}Y18-T4L where the rotamer with the bromine sitting outside [^{mCl}Y18-T4L(o), carbons colored cyan] the loop. Waters are labeled W1-W6, with those that are in positions nearly identical to those of WT* colored and labeled in blue and those in positions unique to the o-rotamer colored and labeled in cyan.

FIG. 5D depicts a stereoimage of the interacting water molecules (labeled W1-W6) in ^{mBr}Y18-T4L. Waters labeled W1 to W6, with those that are in near identical positions relative to WT* colored and labeled in blue, while those in positions unique to the i-rotamer in yellow (along with the carbons of the Y18 side chain).

FIG. 5E depicts a stereoimage of the interacting water molecules (labeled W1-W6) in ^{mBr}Y18-T4L where the rotamer with the bromine sitting outside (^{mBr}Y18-T4L(o), carbons in cyan) the loop. Waters are labeled W1 to W6, with those that are in near identical positions relative to WT* colored and labeled in blue, and those in positions unique to the o-rotamer, colored in cyan.

FIG. 5F depicts a stereoimage of the interacting water molecules (labeled W1-W6) in ^{mI}Y18-T4L. Waters are labeled W2 to W6 (the W1 molecule equivalent to WT* was not observed in this structure), with those that are in near identical positions relative to WT* colored and labeled in blue.

FIG. 6 depicts a graph showing the differences in melting temperatures [ΔT_{M} (▪)] and in melting enthalpies [ΔΔH_{M} (∘)] for ^{mX}Y18-T4L (X═Cl, Br, or I) vs WT* constructs of T4 lysozyme. Standard deviations of the measured values are shown as error bars.

FIG. 7 depicts a graph showing heat capacity (ΔCp) vs hydrophobic solvent accessible surface (% Hydrophobic SAS) at the meta-position of Y18 in the ^{mX}Y18-T4L constructs (where X═H, Cl, Br, or I). A linear regression fit of these data yields the relationship ΔCp=2.31 (% SAS)+2.59 (R²=0.96).

FIG. 8 depicts a graph showing enzymatic activities of each halogenated construct (Cl in diamonds, Br in squares, and I in circles) constructs, as a percent of WT* activity (defined as 100% and indicated by the dashed line) at 23 and 40° C.

FIG. 9 depicts a graph showing percent relative to WT* (100%) as measured at 23° C. (squares) and 40° C. (triangles) vs stability is relative to the ΔG° of WT*. A linear regression fit of the data, excluding ^{mBr}Y18-T4L as the singular outlier at 23° C., results in the relationship % Activity=−91.1 (ΔΔG°)+85.4% (with R²=0.92, solid line).

FIG. 10A depicts a schematic showing how that electrostatic potential (ESP) of 2-halophenol can be calculated as the OH rotates from an angle δ=180° (non-HB trans-OH orientation) to δ=0° (HB cis-OH orientation) in 45° increments.

FIGS. 10B-10D depict QM-calculated ESP maps from +40 kcal/mol to −40 kcal/mol of interaction energy to a positive point charge, reflecting a surface charge that ranges from positive (blue) to negative (red) on the halogen surface where the halogen is Cl (FIG. 10B), Br (FIG. 10C), or I (FIG. 10D).

FIG. 11 depicts an image showing the quantum mechanics energies calculated at the MP2 level (E_{MP2}) for complexes of N-methylacetamide (NMA) with chlorobenzene (left) or 2-chlorophenol (right).

FIG. 12 depicts an image showing the quantum mechanics energies (E_{MP2}) calculated for the ternary complex of the i-Rotamer (blue boxes) and o-Rotamer (red boxes) forms of the ^{mCl}Y18-T4L construct.

FIG. 13A depicts the molecular structure of KIX, the binding protein of the cAMP response element-binding (CREB) transcription factor.

FIG. 13B depicts the hydrogen bond (H-bond) from the hydroxyl group (red) of tyrosine-66 (Y66) to the backbone polypeptide oxygen of the glutamate-16 (E16) residue of the wild-type enzyme.

FIG. 13C depicts a model of the hydrogen bond-enhanced halogen bond (HeX-bond) from the chlorine (green) of the engineered meta-chlorotyrosine.

FIG. 14 is a graph showing the thermal melting of wild-type KIX (WT KIX) and the halogenated constructs determined by differential scanning calorimetry (DSC). The DSC melting curves are shown with background subtracted: WT KIX (black), meta-chloro-Tyr-KIX (clY KIX, green), meta-iodoTyr-KIX (iY KIX, purple).

### DETAILED DESCRIPTION OF THE INVENTION

The present disclosure is based in part on the surprising discovery by the inventors that halogenation of an amino acid residue in a protein increases stability and activity of the protein due to a previously uncharacterized synergistic interaction between the engineered halogen bond (XB) and an intramolecular hydrogen bond (HB), forming a HB enhanced XB (HeX-B) interaction. Accordingly, the present disclosure provides a method of increasing stability of an engineered protein by formation of a HeX-B. It has been surprisingly found that modifying a protein to form a HeX-B stabilizes a protein by more than 1 kcal/mol. Use of the method as described herein was also found to improve thermal stability and increase enzymatic activity in protein engineered to encompass a HeX-B interaction. The methods for engineering a protein to encompass a HeX-B interaction can be used for a number of different applications.

Unless otherwise required by context, singular terms as used herein and in the claims shall include pluralities and plural terms shall include the singular. For example, reference to “a protein” includes a plurality of such proteins and reference to “the protein” includes reference to one or more protein known to those skilled in the art, and so forth.

The use of “or” means “and/or” unless stated otherwise. Furthermore, the use of the term “including,” as well as other forms, such as “includes” and “included,” is not limiting. Also, terms such as “element” or “component” encompass both elements and components comprising one unit and elements and components that comprise more than one subunit unless specifically stated otherwise.

Described herein are several definitions. Such definitions are meant to encompass grammatical equivalents.

The term “halogenation” refers to a chemical reaction that involves the addition of one or more halogen atoms to an amino acid residue.

The term “native” or “native state” when referring to a “native protein”, “native protein function”, and the like refers to the state of a protein in the context of a multicellular organism or in a natural environment.

The term “parent protein” as used herein refers to a protein (native or otherwise) prior to being subjected to manipulation and/or modification to form a halogenated engineered protein. In some instances, a “parent protein” may be a native protein. In other instances, a “parent protein” may be a native protein that has been subjected to artificial manipulation and/or modification prior to halogenation.

The term “engineered protein” refers to a protein that has been artificially manipulated and/or modified in some manner but still maintains the overall global three-dimensional structure (fold) of the parent protein. Non-limiting examples of methods used to generate engineered proteins include chemical manipulation of a parent protein and/or genetic chemical manipulation of a parent protein.

The term “stabilize” or “stability” when referring to “stability of an engineered protein”, “stabilizes the engineered protein”, and the like refers to a protein that maintains the overall native and/or parent folded conformation over a denatured (unfolded or extended) state in any given environment.

### I. Engineered Proteins Including at Least One Halogenated Amino Acid Residue

The present disclosure provides compositions encompassing an engineered protein that includes at least one halogenated amino acid residue, wherein the halogenated amino acid residue may form a hydrogen bond-enhanced halogen bond (HeX-B) which stabilizes the engineered protein. The engineered proteins as described herein can have enhanced structural and/or functional properties. The present disclosure provides an engineered protein with increased thermal stability. The engineered protein as described herein may have increased activity as compared to a parent protein.

In various embodiments, an engineered protein as disclosed herein may comprise any naturally or non-naturally occurring macromolecule. In some aspects, an occurring the macromolecule can be a protein, peptide, or polypeptide. In another aspect, the macromolecule is a protein.

In various embodiments, the protein comprises at least one pocket. As used herein, the term “pocket” refers to a protein having a cavity on its surface, a cavity in its interior, a groove, a cleft, or a combination thereof. In some aspects, the pocket encompasses a hydrophobic region. In other aspects, the hydrophobic region may be completely exposed, partially exposed, or completely not exposed to a solvent. The pocket may be naturally occurring in the parent protein or may be introduced into a parent protein. The pocket of a parent protein may be enhanced in order to better accommodate the addition of a halogen.

The halogenation of one or more amino acids of the parent protein can occur either during synthesis/production of the engineered protein or after synthesis/production of the parent protein. For example, in some embodiments, an engineered protein as disclosed may be formed by introducing a halogenated amino acid into the protein during synthesis/production. In other embodiments, an engineered protein may be formed by chemically modifying a parent protein.

In various embodiments, an engineered protein maybe formed by introducing a halogenated amino acid into the protein during synthesis/production. By way of example, during expression of protein in a bacteria or other cell culture system, halogenated amino acids can be added such they are incorporated into the protein during synthesis/production. Methods of halogenating amino acids are known by those of skill in the art, including those discussed below.

In some embodiments, an engineered protein may be formed by subjecting a parent protein to halogenation (alternatively referred to herein as a “halogenated engineered protein”). In some aspects, one or more fluoride atoms, chlorine atoms, bromine atoms, iodine atoms, or a combination thereof can be added to at least one amino acid residue in an engineered protein as disclosed herein. In some aspects, an engineered protein as disclosed herein may be formed by subjecting a parent protein to free radical halogenation, ketone halogenation, electrophilic halogenation, halogen addition reaction, or a combination thereof. In other aspects, an engineered protein as disclosed herein may be formed by subjecting a parent protein to fluorination, chlorination, bromination, iodination, or a combination thereof.

In various embodiments, an engineered protein as disclosed herein may have at least one, at least 2, at least 3, at least 4, or at least 5 halogenated amino acid residues. As used herein, “amino acids” are represented by their full name, their three letter code, or their one letter code as well known in the art.

An amino acid as disclosed herein may be naturally occurring. A “naturally occurring amino acid” can also be referred to as a “standard amino acid.” Naturally occurring amino acid residues are abbreviated as follows: Phenylalanine is Phe or F; Leucine is Leu or L; Isoleucine is Ile or I; Methionine is Met or M; Valine is Val or V; Serine is Ser or S; Proline is Pro or P; Threonine is Thr or T; Alanine is Ala or A; Tyrosine is Tyr or Y; Histidine is His or H; Glutamine is Gln or Q; Asparagine is Asn or N; Lysine is Lys or K; Aspartic Acid is Asp or D; Glutamic Acid is Glu or E; Cysteine is Cys or C; Tryptophan is Trp or W; Arginine is Arg or R; and Glycine is Gly or G.

An amino acid as disclosed herein may be non-naturally occurring. As used herein a “non-naturally occurring amino acid” refers to any amino acid, modified amino acid, or amino acid analog other than the standard amino acids listed above. In some aspects, a non-naturally occurring amino acid may have side chain groups that distinguish them from a standard amino acid. For example, a non-naturally occurring amino acid may have a side chain group comprising an alkyl-, aryl-, acyl-, keto-, azido-, hydroxyl-, hydrazine, cyano-, halo-, hydrazide, alkenyl, alkynl, ether, thiol, seleno-, sulfonyl-, borate, boronate, phospho, phosphono, phosphine, heterocyclic, enone, imine, aldehyde, ester, thioacid, hydroxylamine, amino group, or the like or any combination thereof. Other examples of non-naturally occurring amino acids can include, but are not limited to, amino acids comprising a photoactivatable cross-linker, spin-labeled amino acids, fluorescent amino acids, metal binding amino acids, metal-containing amino acids, radioactive amino acids, amino acids with novel functional groups, amino acids that covalently or noncovalently interact with other molecules, photocaged and/or photoisomerizable amino acids, amino acids comprising biotin or a biotin analogue, glycosylated amino acids such as a sugar substituted serine, other carbohydrate modified amino acids, keto containing amino acids, amino acids comprising polyethylene glycol or polyether, heavy atom substituted amino acids, chemically cleavable and/or photocleavable amino acids, amino acids with an elongated side chains as compared to natural amino acids, e.g., polyethers or long chain hydrocarbons, e.g., greater than about 5 or greater than about 10 carbons, carbon-linked sugar-containing amino acids, redox-active amino acids, amino thioacid containing amino acids, and amino acids comprising one or more toxic moiety.

In some aspects, a non-naturally occurring amino acid may be an aromatic amino acid with a side chain halo group. In other aspects, a non-naturally occurring amino acid may be a para-substituted aromatic amino acid, an ortho-substituted aromatic amino acid, or a meta substituted aromatic amino acid wherein the substituted aromatic amino acid comprises a halogen selected from chlorine, bromine, fluorine, or iodine. In still other aspects, a non-naturally occurring amino acid may be a para-substituted tyrosine, an ortho-substituted tyrosine, or a meta substituted tyrosine wherein the substituted tyrosine comprises a halogen. In some aspects, a non-naturally occurring amino acid may be 3-chloro-I-tyrosine, 3-bromo-I-tyrosine, or 3-iodo-I-tyrosine.

In some aspects, at least one, at least 2, at least 3, at least 4, or at least 5 standard amino acid residues of a parent protein can be halogenated. In other aspects, at least one, at least 2, at least 3, at least 4, or at least 5 aromatic amino acid residues of a parent protein can be halogenated.

In still other aspects, an amino acid residue that can be halogenated by methods disclosed herein may be a phenylalanine, a tryptophan, a histidine, or a tyrosine. In another aspect, an amino acid residue that can be halogenated by methods disclosed herein is a tyrosine.

In various embodiments, at least one, at least 2, at least 3, at least 4, or at least 5 of amino acid residues halogenated by methods disclosed herein are located in a pocket of a parent protein. In some aspects, at least one halogenated amino acid residue located in a pocket of an engineered protein may be phenylalanine, tryptophan, histidine, or tyrosine. In another aspect, at least one halogenated amino acid residue located in a pocket of an engineered protein is tyrosine.

In some embodiments, the parent protein may be engineered to create or enhance a pocket that is suitable for the addition of a halogen. The halogen may be fully or partially located in this pocket.

In other embodiments, at least one, at least 2, at least 3, at least 4, or at least 5 of amino acid residues halogenated by methods disclosed herein are located approximate to a pocket of the parent protein. In some aspects, at least one halogenated amino acid residue that can be located approximate to a pocket of an engineered protein may be phenylalanine, tryptophan, histidine, or tyrosine. In another aspect, at least one halogenated amino acid residue located approximate to a pocket of an engineered protein is tyrosine.

In various embodiments, halogenation of a parent protein may add a halogen atom to an amino acid residue in the meta-position. In some aspects, halogenation of a parent protein may add a halogen atom to an aromatic amino acid residue in the meta-position. In other aspects, halogenation of a parent protein may add a halogen atom in the meta-position at the 1 position on an aromatic amino acid residue. In still other aspects, halogenation of a parent protein may add a halogen atom in the meta-position at the 3 position on an aromatic amino acid residue.

In various embodiments, a halogenated residue in an engineered protein may be completely exposed to solvent, partially exposed to solvent, or not exposed to solvent. In some aspects, about 1% to about 50% of a halogenated residue in an engineered protein may be exposed to solvent. In other aspects, about 1%, about 5%, about 10%, about 20%, about 30%, about 40%, or about 50% of a halogenated residue in an engineered protein may be exposed to solvent.

In some embodiments, the added halogen is located inside a pocket (i-rotamer) or outside the pocket (o-rotamer). Halogens located inside the pocket may have lower or no exposure to the solvent as compared to halogens outside of the pocket, which may have higher exposure to the solvent. In some embodiments, the percentage of i-rotamer may be at least 40%, at least 50%, at least 60%, at least 70% at least 80%, or at least 90%. In some embodiments, the percentage of i-rotamer may is about 50% or greater. In other embodiments the percentage of i-rotamer may be more than the percentage of o-rotamer. In further embodiments the amount of i-rotamer is about 1% or more greater than the o-rotamer, about 2% or more greater than the o-rotamer, about 3% or more greater than o-rotamer, about 4% or more greater than the o-rotamer, or about 5% or more greater than the o-rotamer. In further embodiments the amount of i-rotamer is about 5% or more greater than the o-rotamer, about 10% or more greater than the o-rotamer, about 15% or more greater than the o-rotamer, about 20% or more greater than the o-rotamer, about 25% or more greater than the o-rotamer, about 30% or more greater than the o-rotamer, about 35% or more greater than o-rotamer, about 40% or more greater than the o-rotamer, about 45% or more greater than o-rotamer, about 50% or more greater than the o-rotamer, about 55% or more greater than the o-rotamer, about 60% or more greater than the o-rotamer, about 65% or more greater than the o-rotamer, about 70% or more greater than the o-rotamer, about 75% or more greater than the o-rotamer, about 80% or more greater than the o-rotamer, about 85% or more greater than the o-rotamer, about 90% or more greater than the o-rotamer, or about 95% or more greater than the o-rotamer.

In various embodiments, halogenation of an amino acid residue in a parent protein may form a halogen bond (XB) in the resulting engineered protein. In some aspects, halogenation of an amino acid residue in a pocket of a parent protein may form a XB in the resulting engineered protein. In other aspects, a halogenated amino acid residue in an engineered protein may form a XB with a non-halogenated amino acid residue in said engineered protein. In still other aspects, a halogenated amino acid residue in a pocket of an engineered protein may form a XB with a non-halogenated amino acid residue in a pocket of said engineered protein. In yet other aspects, a halogenated amino acid residue can form a XB with the backbone carbonyl oxygen of a non-halogenated amino acid residue. In some other aspects, a halogenated amino acid residue can form a XB with a side chain group of a non-halogenated amino acid residue. Non-limiting examples of XBs that can be formed involving side chain groups include: 1) XB with hydroxyls in serine, threonine, and tyrosine; 2) XB with carboxylate groups in aspartate and glutamate, 3) XB with sulfurs in cysteine and methionine; 4) XB with nitrogens in histidine; and 5) XB with the π surfaces of phenylalanine, tyrosine, histidine, and tryptophan.

In some embodiments, a halogenated amino acid residue in an engineered protein may form a XB with any amino acid residue located at about 2.0 Å to about 5.0 Å distance from the halogenated amino acid residue. In other embodiments, a halogenated amino acid residue in an engineered protein may form a XB with any amino acid residue located at about 2.0 Å, about 2.5 Å, about 3.0 Å, about 3.5 Å, about 4.0 Å, about 4.5 Å, or about 5.0 Å distance from the halogenated amino acid residue.

In various embodiments, a XB between a halogenated amino acid residue and a non-halogenated amino acid residue in an engineered protein can form an electropositive σ-hole. An σ-hole can result from redistribution of the valence electron in the p_{z}-atomic orbital of the halogen to participate in XB formation, leaving a hole that partially exposes the positive nuclear charge. In various embodiments, a XB between a halogenated amino acid residue and a non-halogenated amino acid residue in an engineered protein can form an electronegative annulus around the center of the XB.

In various embodiments, a XB between a halogenated amino acid residue and a non-halogenated amino acid residue in an engineered protein can interact with at least one intramolecular hydrogen bond (HB). As used herein, the term “intramolecular bond” refers to a bond existing and/or taking place within a protein.

In various embodiments, a HB interacting between a XB in an engineered protein disclosed herein can intensify the electropositive σ-hole. In some aspects, a HB interacting between a XB in an engineered protein disclosed herein can intensify the electropositive σ-hole by about 2-fold to about 1,000-fold. In other aspects, a HB interacting between a XB in an engineered protein disclosed herein can intensify the electropositive σ-hole by 2-fold, by about 3-fold, by about 4-fold, by about 5-fold, by about 6-fold, by about 7-fold, by about 8-fold, by about 9-fold, by about 10-fold, by about 20-fold, by about 50-fold, by about 100-fold, by about 500-fold, or by about 1,000-fold.

In various embodiments, interaction between a XB and a HB in an engineered protein disclosed herein can increase the strength of the XB. In various aspects, an interaction between a XB and a HB in an engineered protein disclosed herein can increase the strength of the XB in an additive manner. In other aspects, an interaction between a XB and a HB in an engineered protein disclosed herein can increase the strength of the XB in a synergistic manner. In some aspects, interaction between a XB and a HB in an engineered protein disclosed herein can increase the strength of the XB by about 2-fold to about 1,000-fold. In other aspects, interaction between a XB and a HB in an engineered protein disclosed herein can increase the strength of the XB by about 2-fold, by about 3-fold, by about 4-fold, by about 5-fold, by about 6-fold, by about 7-fold, by about 8-fold, by about 9-fold, by about 10-fold by about 20-fold, by about 50-fold, by about 100-fold, by about 500-fold, or by about 1,000-fold.

As used herein, where an interaction between an engineered XB and an intramolecular HB increases the strength of the XB, the interaction is referred to as a “HB enhanced XB interaction” or “HeX-B.”

In various embodiments, an engineered protein disclosed herein encompassing a HeX-B can be more stable than a parent protein under the same conditions. Use of the term “stable” when referring to a protein disclosed herein is referring to a protein's ability to retain its conformation (in some cases native) as required for normal protein function. In some aspects, an engineered protein disclosed herein encompassing a HeX-B can be about 5%, about 10%, about 25%, about 50%, about 75%, about 100%, about 200%, about 500%, or about 1,000% more stable than a parent protein under the same conditions.

In various embodiments, an engineered protein disclosed herein encompassing a HeX-B can be more conformationally rigid than a parent protein under the same conditions. Use of the term “conformationally rigid” when referring to a protein disclosed herein is referring to a protein's ability to kept its substructures fixed into a functional conformation. In some instances, the functional conformation is similar or identical to the native conformation. In other instances the functional conformation is similar or identical to the parent conformation. In some aspects, an engineered protein disclosed herein encompassing a HeX-B can be about 5%, about 10%, about 25%, about 50%, or about 75% more conformationally rigid than a parent protein under the same conditions.

In some aspects, an entropy of unfolding at melting temperature (ΔS_{M}) for an engineered protein disclosed herein may be about 1 cal mol⁻¹ K⁻¹ to about 15 cal mol⁻¹ K⁻¹ higher than a parent protein under the same conditions. In other aspects, an entropy of unfolding at melting temperature (ΔS_{M}) for an engineered protein disclosed herein may be about 1 cal mol⁻¹ K⁻¹, about 2 cal mol⁻¹ K⁻¹, about 3 cal mol⁻¹ K⁻¹, about 4 cal mol⁻¹ K⁻¹, about 5 cal mol⁻¹ K⁻¹, about 6 cal mol⁻¹ K⁻¹, about 7 cal mol⁻¹ K⁻¹, about 8 cal mol⁻¹ K⁻¹, about 9 cal mol⁻¹ K⁻¹, about 10 cal mol⁻¹ K⁻¹, about 11 cal mol⁻¹ K⁻¹, about 12 cal mol⁻¹ K⁻¹, about 13 cal mol⁻¹ K⁻¹, about 14 cal mol⁻¹ K⁻¹, or about 15 cal mol⁻¹ K⁻¹ higher than a parent protein under the same conditions.

In various embodiments, an engineered protein disclosed herein encompassing a HeX-B can have higher thermal stability compared to a parent protein under the same conditions. Use of the term “thermal stability” when referring to a protein disclosed herein refers to a protein's ability to resist to changes its protein structure due to applied heat.

In some embodiments, thermal stability can be determined based on melting point. In some aspects, an engineered protein disclosed herein encompassing a HeX-B can have a higher melting temperature (T_{M}) compared to a parent protein. In some aspects, the T_{M} of an engineered protein disclosed herein encompassing a HeX-B can be about 1% to about 50% higher than a parent protein under the same conditions. In other aspects, the T_{M} of an engineered protein disclosed herein encompassing a HeX-B can be about 1%, about 5%, about 10%, about 25%, about 50% higher than a parent protein under the same conditions. In still other aspects, the T_{M} of an engineered protein disclosed herein encompassing a HeX-B can be about 0.5° C. to about 10° C. higher than a parent protein under the same conditions. In yet other aspects, the T_{M} of an engineered protein disclosed herein encompassing a HeX-B can be about 1° C., about 2° C., about 3° C., about 4° C., about 5° C., about 6° C., about 7° C., about 8° C., about 9° C., or about 10° C. higher than a parent protein under the same conditions.

In other embodiments, thermal stability can be determined based on melting entropy. In some aspects, an engineered protein disclosed herein encompassing a HeX-B can have a higher enthalpy of melting (ΔH_{M}) compared to a parent protein. In some aspects, the ΔH_{M} of an engineered protein disclosed herein encompassing a HeX-B can be about 1% to about 50% higher than a parent protein under the same conditions. In other aspects, the ΔH_{M} of an engineered protein disclosed herein encompassing a HeX-B can be about 1%, about 5%, about 10%, about 25%, about 50% higher than a parent protein under the same conditions. In some aspects, the ΔH_{M} of an engineered protein disclosed herein encompassing a HeX-B can be about 1.0 kcal/mol can be more than 1 kcal/mol higher than a parent protein under the same conditions. In still other aspects, the ΔH_{M} of an engineered protein disclosed herein encompassing a HeX-B can be about 1.0 kcal/mol to about 20.0 kcal/mol higher than a parent protein under the same conditions. In other aspects, the ΔH_{M} of an engineered protein disclosed herein encompassing a HeX-B can be about 1.0 kcal/mol, about 2.0 kcal/mol, about 5.0 kcal/mol, about 10.0 kcal/mol, about 15 kcal/mol, or about 20.0 kcal/mol. In yet other aspects, the ΔH_{M} of an engineered protein disclosed herein encompassing a HeX-B can be about 1.0 kcal/mol, about 1.5 kcal/mol, about 2.0 kcal/mol, about 2.5 kcal/mol, about 3.0 kcal/mol, about 3.5 kcal/mol, about 4.0 kcal/mol, about 4.5 kcal/mol, 5.0 kcal/mol, about 5.5 kcal/mol, 6.0 kcal/mol, about 6.5 kcal/mol, 7.0 kcal/mol, about 7.5 kcal/mol, or about 8.0 kcal/mol higher than a parent protein under the same conditions.

An engineered protein disclosed herein encompassing a HeX-B can be a protein may be a fibrous protein, a globular protein, or a membrane protein. In other aspects, a protein can be an enzyme, a structural protein, a signaling protein, a regulatory protein, a transport protein, a sensory protein, a motor protein, a defense protein, a hormonal protein, or a storage protein. In still other aspects, a engineered protein disclosed herein encompassing a HeX-B may contribute to a physiological process, a biological process, a cellular process, a cellular physiological process, catalytic activity, aromatase activity, motor activity, helicase activity, integrase activity, antioxidant activity, metabolism, macromolecule metabolism, proteolysis, amino acid and derivative metabolism, nucleobase, nucleoside, nucleotide and nucleic acid metabolism, biosynthesis, catabolism, kinase activity, oxidoreductase activity, transferase activity, hydrolase activity, lyase activity, isomerase activity, ligase activity, enzyme regulator activity, signal transducer activity, structural molecule activity, cytoskeleton, extracellular matrix, binding, receptor activity, protein binding, lipid binding, cell motility, membrane fusion, cell communication, regulation of biological process, development, cell differentiation, response to stimulus, behavior, cell adhesion, cell death, transport, protein transporter activity, nuclear transport, ion transporter activity, channel or pore class transporter activity, carrier activity, permease activity, secretion, electron transporter activity, electron transport, pathogenesis, chaperone regulator activity, nucleic acid binding, transcription regulator activity, extracellular structure organization and biogenesis, translation regulator activity, or a combination thereof.

In another aspect, the engineered protein disclosed herein encompassing a HeX-B is an enzyme. In some aspects, an enzyme may be an amylase, a protease, or a lipase. In other aspects, an amylase may be an α-amylase, a β-amylase, or a γ-amylase. In still other aspects, a protease may be serine protease, a cysteine protease, a threonine protease, an aspartic protease, a glutamic protease, or a metalloprotease. In yet other aspects, a lipase may be a bile salt-dependent lipase, a lysosomal lipase, a hormone-sensitive lipase, a gastric lipase, a lingual lipase, a pancreatic lipase, a hepatic lipase, an endothelial lipase, or a lipoprotein lipase.

In various embodiments, an engineered protein disclosed herein encompassing a HeX-B can be an enzyme. In some aspects, an engineered enzyme disclosed herein encompassing a HeX-B can have higher enzymatic activity compared to a parent enzyme under the same conditions. In some aspects, an engineered enzyme disclosed herein encompassing a HeX-B can have about 2%, about 5%, about 10%, about 25%, about 50%, or about 75% higher enzymatic activity compared to a parent enzyme under the same conditions.

In another aspect, the engineered protein disclosed herein encompassing a HeX-B is a transcription factor. In some aspects, a transcription factor may be constitutively active, conditionally active, developmental, extracellular ligand signal-dependent, intracellular ligand signal-dependent, cell membrane receptor-dependent signal-dependent, resident nuclear factor signal-dependent, or latent cytoplasmic factor signal-dependent. In other aspects, a transcription factor may be selected from the zinc-coordinating DNA-binding domains superclass, the helix-turn-helix superclass, the beta-scaffold factors with minor groove contacts superclass, or the other transcription factors superclass. In still other aspects, a transcription factor may be selected from the leucine zipper factor class, the helix-loop-helix factor class, the helix-loop-helix/leucine zipper factor class, the NF-1 class, the RF-X class, the bHSH class, the Cys4 zinc finger of nuclear receptor type class, the diverse Cys4 zinc finger class, the Cys2His2 zinc finger domain class, the Cys6 cysteine-zinc cluster class, the zinc fingers of alternating composition class, the homo domain class, the paired box class, the Fork head/winged helix class, the heat shock factor class, the Tryptophan cluster class, the A (transcriptional enhancer factor) domain class, the RHR (Rel homology region) class, the STAT class, the p53 class, the MADS box class, the beta-Barrel alpha-helix transcription factor class, the TATA binding pair class, the HMG-box class, the heteromeric CCAAT factor class, the grainyhead class, the cold-shockdomain factor class, the runt class, the copper first protein class, the HMGI(Y) class, pocket domain class, the E1A-like factor class, or the AP2/EREBP-related factor class.

In various embodiments, an engineered protein disclosed herein encompassing a HeX-B can be a transcription factor. In some aspects, an engineered transcription factor disclosed herein encompassing a HeX-B can have higher transcriptional activity compared to a parent transcription factor under the same conditions. In other aspects, an engineered transcription factor disclosed herein encompassing a HeX-B can have about 2%, about 5%, about 10%, about 25%, about 50%, or about 75% higher transcriptional activity compared to a parent transcription factor under the same conditions. In still other aspects, an engineered transcription factor disclosed herein encompassing a HeX-B can have lower transcriptional activity compared to a parent transcription factor under the same conditions. In some aspects, an engineered transcription factor disclosed herein encompassing a HeX-B can have about 2%, about 5%, about 10%, about 25%, about 50%, or about 75% lower transcriptional activity compared to a parent transcription factor under the same conditions. In other aspects, an engineered transcription factor disclosed herein encompassing a HeX-B can block transcriptional activity.

### II. Methods of Making Engineered Proteins Including at Least One Halogenated Amino Acid Residue

The present disclosure provides methods of making an engineered protein that includes at least one halogenated amino acid residue, wherein the halogenated amino acid residue may form a hydrogen bond-enhanced halogen bond (HeX-B) which stabilizes the engineered protein. In various embodiments, methods of making an engineered protein comprises obtaining a parent protein and subjecting a parent protein to genetic modification, chemical modification, or both. In some embodiments, methods of making an engineered protein include subjecting a parent protein to halogenation. In further embodiments, methods of making an engineered protein include selecting at least one amino acid on a parent protein for halogenation.

The present disclosure also provides methods of increasing the stability of a protein through formation of a hydrogen bond-enhanced halogen bond (HeX-B) by halogenating at least one amino acid residue of the protein. The methods as described herein may also increase thermal stability of an engineered protein as compared to a parent protein. The methods as described herein may also increase activity of an engineered protein as compared to a parent protein.

The methods of stabilizing proteins as described herein can provide a unique strategy for the design of a number of engineered proteins with enhanced structural and/or functional properties.

(a) Methods of Generating a Parent Protein.

In various embodiments, methods of making an engineered protein include obtaining a parent protein. In various embodiments, a parent protein can be obtained by isolating from a native source. In some aspects, a parent protein can be obtained by isolating from animal, cellular and/or serum sources. In some aspects, a parent protein as disclosed herein can be isolated from a subject's organs, tissues, cells, blood, or a combination thereof. The term “subject” refers to an animal, including but not limited to a mammal including a human and a non-human primate (for example, a monkey or great ape), a cow, a pig, a cat, a dog, a rat, a mouse, a horse, a goat, a rabbit, a sheep, a hamster, a guinea pig). In other aspects, a parent protein as disclosed herein can be isolated from a solid tumor and/or primary cell lines derived from a tumor. In other aspects, a parent protein as disclosed herein can be isolated from plant tissues, plant cells, or both. In still other aspects, a parent protein as disclosed herein can be isolated from microorganisms. In yet other aspects, a parent protein as disclosed herein can be isolated from bacteria, fungi, algae, protozoa, or a combination thereof. In still other aspects, a parent protein as disclosed herein can be isolated from primary cell lines derived from insects, vertebrate animals, or plants.

In some embodiments, a method for isolating a parent protein from a native source can be any known in the current art. In some aspects, a method for isolating a parent protein from a native source can be chromatography. In other aspects, a method for isolating a parent protein from a native source can be affinity chromatography, ion exchange chromatography, gel filtration chromatography, or reverse-phase chromatography.

In various embodiments, a parent protein can be a recombinant protein. As used herein, the term “recombinant protein” refers to a protein made from polynucleotides encoding for a protein. In various embodiments, methods of making protein include genetic modification. Methods of genetic modification are known in the art. In some aspects, polynucleotides encoding for a protein can be genetically modified by methods including, but not limited to, PCR, site-directed mutagenesis, site-saturation mutagenesis, DNA shuffling, artificial transcription factors, and/or Multiplex Automated Genome Engineering (MAGE).

In some embodiments, a parent protein may be modified to create or enhance an existing pocket for the halogen. The polynucleotides encoding a parent protein may be genetically modified to create or enhance an existing pocket for the halogen.

In various embodiments, polynucleotides encoding for a parent protein are genetically modified to allow for selective incorporation of at least one non-naturally occurring amino acid during protein synthesis. Methods of genetic modification for selective incorporation of non-naturally occurring amino acids are known in the art. An example of a method, but not limited to, includes development of orthogonal aminoacyl-tRNA synthetase (aaRS)/tRNA pairs wherein the orthogonal aaRS selectively recognizes its cognate orthogonal tRNA over endogenous tRNAs, and the orthogonal tRNA is a substrate for the orthogonal aaRS but a poor substrate for endogenous synthetases. In some aspects, an anticodon in an orthogonal tRNA can be genetically modified to specifically recognize a stop codon. In other aspects, stop codons as used herein may be amber (TAG) codons, ochre (TAA) codons, opal (TGA) codons, or a combination thereof. In still other aspects, an orthogonal aaRS can be generated by genetically modifying polynucleotides encoding for a parent protein to insert a stop codon at, before, and/or after the codon that codes for the amino acid desired to be replaced with a non-naturally occurring amino acid.

The polynucleotides of a parent protein can be incorporated into a vector, which can be introduced into a host cell for expression. Methods of expressing proteins in host cells are known in the art. By way of non-limiting examples, a recombinant protein as disclosed herein can be expressed in a mammalian cell-based protein expression, an insect cell-based protein expression, a yeast cell-based protein expression, a bacterial cell-based protein expression, an algal cell-based protein expression, or an in vitro (cell-free) protein expression. In other aspects, a recombinant parent protein as disclosed herein can be expressed in an expression host cell selected from selected from fungal (filamentous fungal or yeast), insect, mammalian animal cells, from transgenic plant cells or from transgenic animals. In some aspects, a host cell can be a mammalian cell, such as an CHO cell, BHK or HEK cell, e.g. HEK293, or an insect cell, such as an SF9 cell, or a yeast cell, e.g. Saccharomyces cerevisiae, Pichia pastoris, or a bacterial cell, e.g., Escherichia coli.

The term “vector”, as used herein, refers to a DNA or RNA molecule such as a plasmid, virus or other vehicle, which contains one or more heterologous or recombinant DNA sequences and is designed for transfer between different host cells. In some aspects, a vector for use herein may be any recombinant vector capable of expression of a protein or polypeptide of interest or a fragment thereof, for example, an adeno-associated virus (AAV) vector, a lentivirus vector, a retrovirus vector, a replication competent adenovirus vector, a replication deficient adenovirus vector (e.g., a gutless adenovirus vector), a herpes virus vector, a baculovirus vector or a nonviral plasmid. In other aspects, vector for recombinant parent protein expression may include any of a number of promoters, wherein the promoter is constitutive, regulatable or inducible, cell type specific, tissue-specific, or species specific. In still other aspects, a vector may further comprise a signal sequence for the coding sequence of a domain of the protein or polypeptide. Non limiting examples of vectors for recombinant parent protein expression can include pALTER, pBAD, pCal, pcDNA, pET, pGEMEX, pGEX, pHAT, pLEX, pMAL, pPro, pQE, pRSET, pSE, pThio, pTrc, and pTriEx. In some aspects, a vector for recombinant parent protein expression can include a poly-histidine (His) tag, a calmodulin binding protein (CBP) tag, a maltose-binding protein (MBP) tag, a glutathione-S transferase (GST) tag, a green fluorescent protein (GFP) tag, a c-Myc tag, a human influenza hemagglutinin (HA) tag, a thioredoxin (TXN) tag, a V5 tag, a FLAG tag, or a combinations thereof. Non-limiting examples of vectors that express synthetase and cognate codon (amber, opal, or ochre) suppressing tRNA include pDule2-pCNF, pMAH-POLY, and pRST11B-AS3_4.

(b) Methods of Producing Engineered Proteins by Halogenation

In various embodiments, methods of making an engineered protein include chemically modifying a parent protein. In some aspects, methods of making an engineered protein by chemically modification of a parent protein can include, but are not limited to, modification of proteins using the reactivity of naturally occurring amino acids, modification by bioorthogonal reactions using unnatural amino acids, most of which can be site-selectively incorporated into proteins-of-interest using genetic codon expansion techniques, and recognition driven methods. In an aspect, a method of chemically modifying a parent protein is halogenation. In some embodiments, methods of making an engineered protein include producing an engineered protein containing at least one halogenated amino acid from a cell culture system.

In various embodiments, engineered proteins can be produced from genetically modified polynucleotides that encode for selective incorporation of at least one non-naturally occurring amino acid into a parent protein during protein synthesis. In some aspects, expression vectors comprising genetically modified parent protein polynucleotides can be introduced into a host cell. In other aspects, expression vectors comprising genetically modified parent protein polynucleotides can be introduced into a host cell in addition to at least one other vector. In still other aspects, expression vectors comprising genetically modified parent protein polynucleotides and a vector that expresses a synthetase and cognate codon suppressing tRNA are transformed into a host cell at the same time.

The transformation of the host cell with a polynucleotide or vector as disclosed herein can be carried out by standard methods, as for instance described in Sambrook and Russell (2001), Molecular Cloning: A Laboratory Manual, CSH Press, Cold Spring Harbor, N.Y., USA; Methods in Yeast Genetics, A Laboratory Course Manual, Cold Spring Harbor Laboratory Press, 1990. In some aspects, a recombinant parent protein as disclosed herein can be expressed in a mammalian cell-based protein expression, an insect cell-based protein expression, a yeast cell-based protein expression, a bacterial cell-based protein expression, an algal cell-based protein expression, or an in vitro (cell-free) protein expression. In other aspects, a recombinant parent protein as disclosed herein can be expressed in an expression host cell selected from selected from fungal (filamentous fungal or yeast), insect, mammalian animal cells, from transgenic plant cells or from transgenic animals. In some aspects, a host cell can be a mammalian cell, such as an CHO cell, BHK or HEK cell, e.g. HEK293, or an insect cell, such as an SF9 cell, or a yeast cell, e.g. Saccharomyces cerevisiae, Pichia pastoris, or a bacterial cell, e.g., Escherichia coli.

In various embodiments, host cells co-transformed with an expression vector comprising genetically modified parent protein polynucleotides and a vector that expresses a synthetase and cognate codon suppressing tRNA are cultured to comprise a cell culture system. In some aspects, a cell culture system comprises nutrient medium meeting the requirements of the particular host cell used, in particular in respect of the pH value, temperature, salt concentration, aeration, antibiotics, vitamins, trace elements etc.

In various embodiments, a cell culture system comprises medium supplemented with at least one non-naturally occurring amino acid. Non-naturally occurring amino acids supplemented in the medium can assimilated by the cells at the genetically modified stop codon in the parent protein. In some aspects, medium can be supplemented with a non-naturally occurring aromatic amino acid with a side chain halo group. In other aspects, medium can be supplemented with a non-naturally occurring para-substituted aromatic amino acid, an ortho-substituted aromatic amino acid, or a meta substituted aromatic amino acid wherein the substituted aromatic amino acid comprises a halogen selected from chlorine, bromine, fluorine, or iodine. In still other aspects, medium can be supplemented with a para-substituted tyrosine, an ortho-substituted tyrosine, or a meta substituted tyrosine wherein the substituted tyrosine comprises a halogen selected from chlorine, bromine, fluorine, or iodine. In some aspects, medium can be supplemented with 3-chloro-I-tyrosine, 3-bromo-I-tyrosine, or 3-iodo-I-tyrosine. Assimilation of a supplemented non-naturally occurring amino acid with a side chain halo group by the cells at the genetically modified stop codon in the parent protein can result in a halogenated amino acid reside.

In some aspects, recombinant parent protein as disclosed herein can be harvested from the cell culture system and purified out by standard methods, as for instance described in Sambrook and Russell (2001), Molecular Cloning: A Laboratory Manual, CSH Press, Cold Spring Harbor, N.Y., USA; Methods in Yeast Genetics, A Laboratory Course Manual, Cold Spring Harbor Laboratory Press, 1990.

In various embodiments, methods of making an engineered protein include halogenation of a parent protein. In some aspects, a method of halogenation may be selected from free radical halogenation, ketone halogenation, electrophilic halogenation, halogen addition reaction, Hunsdiecker reaction, Sandmeyer reaction, Hell-Volhard-Zelinsky halogenation, and oxychlorination. In an aspect, a method of halogenating a parent protein is by electrophilic halogenation. In some aspects, halogenation can be performed in the presence of a Lewis acid. A Lewis acid may be species that can accept a pair of electrons. Non-limiting examples of a Lewis acid include copper (Cu₂), iron (Fe²⁺ and Fe³⁺), hydrogen ion (H⁺), boron trifluoride (BF₃), aluminum fluoride (AlF₃), silicon tetrabromide (SiBr₄), silicon tetrafluoride (SiF₄), carbon dioxide (CO₂), and sulfur dioxide (SO₂).

In some aspects, method of halogenating a parent protein may be enzyme-catalyzed halogenation. In some aspects, enzyme-catalyzed halogenation can be performed in the presence of one or more halogenases. In other aspects, halogenases for halogenation methods disclosed herein may be selected from the heme-dependent haloperoxidase class, the vanadium-dependent haloperoxidase class, the fluorinase class, the non-heme-iron-O₂-dependent halogenase class, or the flavin-dependent halogenase class.

In various embodiments, a method of halogenating a parent protein may be based on the halogen type. In some aspects, a halogen selected for use in methods of halogenation disclosed herein may be from group 17. In other aspects, a halogen selected for use in methods of halogenation disclosed herein may be from group 17. In other aspects, a halogen selected for use in methods of halogenation disclosed herein may be fluorine (F), chlorine (Cl), bromine (Br), iodine (I), astatine (At), or tennessine (Ts). In some aspects, a method of halogenating a parent protein may be fluorination, chlorination, bromination, iodination, or a combination thereof.

In various embodiments, method of halogenating a parent protein as disclosed herein can add a halogen atom to at least 2, at least 3, at least 4, or at least 5 halogenated amino acid residues. In some aspects, methods disclosed herein halogenate at least one aromatic amino acid residues of a parent protein. In other aspects, methods disclosed herein halogenate at least one aromatic amino acid residues of a parent protein selected from a phenylalanine, a tryptophan, a histidine, or a tyrosine. In an aspect, methods disclosed herein halogenate a tyrosine residue of a parent protein.

In various embodiments, methods of halogenating a parent protein as disclosed herein may add a halogen atom to an amino acid residue in the meta-position. In some aspects, methods of halogenation of a parent protein may add a halogen atom to an aromatic amino acid residue in the meta-position. In other aspects, methods of halogenation of a parent protein may add a halogen atom in the meta-position at the 1 position on an aromatic amino acid residue. In still other aspects, methods of halogenation of a parent protein may add a halogen atom in the meta-position at the 3 position on an aromatic amino acid residue.

In various embodiments, methods of halogenating a parent protein as disclosed herein may form a XB in the resulting engineered protein. In some aspects, methods of halogenation as disclosed herein may result in a short C—X ⋅ ⋅ ⋅ O—Y interaction, wherein C—X is a carbon-bonded chlorine, bromine, or iodine, and O—Y is a carbonyl, hydroxyl, charged carboxylate, or phosphate group; X ⋅ ⋅ ⋅ O distance is less than or equal to the sums of the respective van der Waals radii (3.27 Å for Cl ⋅ ⋅ ⋅ 0, 3.37 Å for Br ⋅ ⋅ ⋅ O, and 3.50 Å for I ⋅ ⋅ ⋅ O); and can conform to the geometry seen in small molecules, with the C—X ⋅ ⋅ ⋅ O angle ≈165° (consistent with a strong directional polarization of the halogen) and the X ⋅ ⋅ ⋅ O—Y angle ≈120°. In other aspects, methods of halogenation as disclosed herein may result in one or more alternative geometries, depending on which of the two types of donor systems are involved in the interaction. In assume aspects, a donor systems involved in the interaction may be a lone pair electrons of oxygen (and, to a lesser extent, nitrogen and sulfur) atoms or delocalized π-electrons of peptide bonds or carboxylate or amide groups.

In various embodiments, at least one halogenated amino acid can be selected to be in a pocket of the engineered protein. In other aspects, a halogenated amino acid can be selected to replace an aromatic standard amino acid residue in a protein pocket. In still other aspects, a halogenated amino acid can be selected to replace a tyrosine residue in a protein pocket. In some aspects, a halogenated amino acid is placed in a protein pocket at a position with known interresidue interactions. In other aspects, a halogenated amino acid is placed in a protein pocket with a small void space. In still other aspects, a halogenated amino acid to be placed in a protein pocket with a small void space is selected based on halogen size. In some aspects, a protein pocket with a small void space can accommodate a halogen with a covalent radius less than about 75 pm, about 100, about 120, or about 140. In some aspects, a protein pocket can only accommodate fluorine (covalent radius=71 pm). In other aspects, a protein pocket can only accommodate fluorine and/or chlorine (covalent radius=99 pm). In still other aspects, a protein pocket can only accommodate fluorine, chlorine, and/or bromine (covalent radius=114 pm). In yet other aspects, a protein pocket can only accommodate fluorine, chlorine, bromine and or iodine (covalent radius=133 pm).

In various embodiments, at least one halogenated amino acid can be selected to be in a hydrophobic pocket of the engineered protein. In various embodiments, at least one halogenated amino acid can be selected to be in a hydrophilic pocket of the engineered protein. In various embodiments, at least one halogenated amino acid can be selected to be in a pocket of an engineered protein comprising at least one intact OH substituent. In other various embodiments, at least one halogenated amino acid can be selected to be in a pocket of an engineered protein comprising at least one at least one carbonyl oxygen.

In still other aspects, a halogenated amino acid can be placed in a protein pocket with a small void space capable of forming biological XBs. In other aspects, a halogenated amino acid can be placed in a protein pocket with a small void space capable of forming biological XBs. In yet other aspects, a halogenated amino acid can be placed in a protein pocket with a small void space capable of forming a biological XB to at least one carbonyl oxygen. In other aspects, a halogenated amino acid can be placed in a protein pocket with a small void space comprising an intermolecular HB and is capable of forming a biological XB to at least one carbonyl oxygen. In some other aspects, a halogenated amino acid can be placed in a protein pocket with a small void space that can accommodate at least one halogen to form an XB to a carbonyl oxygen in a geometry that is perpendicular to a intermolecular HB. In some other aspects, a halogenated amino acid can be placed in a protein pocket with a small void space that can accommodate at least one halogen to form an XB with a strength of about 5 kJ/mol to about 180 kJ/mol.

In some embodiments, a halogenated amino acid residue can be placed in a protein pocket to form a XB with any amino acid residue located at about 2.0 Å to about 5.0 Å distance from the halogenated amino acid residue. In some embodiments, a halogenated amino acid residue can be placed in a protein pocket to form a XB with any amino acid residue located at about 2.0 Å, about 2.5 Å, about 3.0 Å, about 3.5 Å, about 4.0 Å, about 4.5 Å, or about 5.0 Å distance from the halogenated amino acid residue.

In other aspects, a halogenated amino acid can be placed in a protein pocket with a small void space comprising an intermolecular HB forming a XB to at least one carbonyl oxygen. In some aspects, XB interaction with an HB anisotropically distributes electron density in the halogen atom. In other aspects, a halogenated amino acid can be placed in a protein pocket to generate a XB with a region of higher electron density to form a belt orthogonal to the covalent bond with a lower region of lower electron density. In some aspects, a halogenated amino acid can be placed in a protein pocket to generate a XB with depleted electron density on the elongation of the covalent bond to form attractive interactions with electron-rich sites.

In various embodiments, methods of placing a halogenated amino acid can in a protein pocket disclosed herein comprises a HB interaction between a XB wherein HB can intensify the electropositive σ-hole. In some aspects, a halogenated amino acid placed in a protein pocket to generate a HB interaction between a XB can intensify the electropositive σ-hole by about 2-fold to about 10-fold. In other aspects, a halogenated amino acid placed in a protein pocket to generate a HB interaction between a XB can intensify the electropositive σ-hole by 2-fold, by about 3-fold, by about 4-fold, by about 5-fold, by about 6-fold, by about 7-fold, by about 8-fold, by about 9-fold, by about 10-fold, by about 20-fold, by about 50-fold, by about 100-fold, by about 500-fold, or by about 1,000-fold.

In various embodiments, a halogenated amino acid can be placed in a protein pocket with a small void space comprising to generate an interaction between a XB and a HB in an engineered protein disclosed wherein the interaction can increase the strength of the XB. In various aspects, a halogenated amino acid can be placed in a protein pocket to increase the strength of the XB in an additive manner. In other aspects, a halogenated amino acid can be placed in a protein pocket to increase the strength of the XB in a synergistic manner. In some aspects, a halogenated amino acid can be placed in a protein pocket to increase the strength of the XB by about 2-fold to about 1,000-fold. In other aspects, interaction between a XB and a HB in an engineered protein disclosed herein can increase the strength of the XB by about 2-fold, by about 3-fold, by about 4-fold, by about 5-fold, by about 6-fold, by about 7-fold, by about 8-fold, by about 9-fold, by about 10-fold by about 20-fold, by about 50-fold, by about 100-fold, by about 500-fold, or by about 1,000-fold.

In various embodiments, at least one halogenated amino acid can be rotated to be outside the pocket (o-rotamer) of the engineered protein. In other various embodiments, at least one halogenated amino acid can be rotated to be inside the pocket (i-rotamer) of the engineered protein. In some aspects, the size of a halogen can be used to predict its rotation. In some aspects, i-rotamer propensity increases as the halogen becomes smaller. In other aspects, at least one halogenated amino acid can be rotated to be inside the pocket (i-rotamer) of the engineered protein to reduce solvent exposure. In some aspects, at least one halogenated amino acid can be rotated to be inside the pocket wherein only about 12% to about 25% of the halogenated amino acid is solvent accessible.

In various embodiments, methods of placing a halogenated amino acid in a protein pocket disclosed herein can generate a HeX-B. In some aspects, a halogenated amino acid can be placed in a protein pocket to stabilize an engineered protein disclosed herein encompassing a HeX-B more than a parent protein under the same conditions. In some aspects, selection of a halogenated amino acid to include in an engineered protein disclosed herein can result in an engineered protein that is about 5%, about 10%, about 25%, about 50%, about 75%, about 100%, about 200%, about 500%, or about 1,000% more stable than a parent protein under the same conditions.

In various embodiments, methods of placing a halogenated amino acid in a protein pocket disclosed herein can generate a more conformationally rigid engineered protein than a parent protein under the same conditions. Selection of a halogenated amino acid to include in an engineered protein disclosed herein can result in an engineered protein that is about 5%, about 10%, about 25%, about 50%, or about 75% more conformationally rigid than a parent protein under the same conditions.

In various embodiments, methods of placing a halogenated amino acid in a protein pocket disclosed herein may increase the entropy of unfolding at melting temperature (ΔS_{M}) for an engineered protein disclosed herein may be about 1 cal mol⁻¹ K⁻¹ to about 15 cal mol⁻¹ K⁻¹ higher than a parent protein under the same conditions. In other aspects, an entropy of unfolding at melting temperature (ΔS_{M}) for an engineered protein disclosed herein may be about 1 cal mol⁻¹ K⁻¹, about 2 cal mol⁻¹ K⁻¹, about 3 cal mol⁻¹ K⁻¹, about 4 cal mol⁻¹ K⁻¹, about 5 cal mol⁻¹ K⁻¹, about 6 cal mol⁻¹ K⁻¹, about 7 cal mol⁻¹ K⁻¹, about 8 cal mol⁻¹ K⁻¹, about 9 cal mol⁻¹ K⁻¹, about 10 cal mol⁻¹ K⁻¹, about 11 cal mol⁻¹ K⁻¹, about 12 cal mol⁻¹ K⁻¹, about 13 cal mol⁻¹ K⁻¹, about 14 cal mol⁻¹ K⁻¹, or about 15 cal mol⁻¹ K⁻¹ higher than a parent protein under the same conditions.

In various embodiments, methods of placing a halogenated amino acid in a protein pocket disclosed herein may increase an engineered protein's thermal stability compared to a parent protein under the same conditions.

In some embodiments, thermal stability can be determined based on melting point. Methods of placing a halogenated amino acid in a protein pocket disclosed herein may increase an engineered protein's melting temperature (T_{M}) compared to a parent protein. In some aspects, methods of generating an engineered protein as disclosed herein may increase the T_{M} of the engineered protein to about 1% to about 50% higher than a parent protein under the same conditions. In other aspects, the T_{M} of an engineered protein disclosed herein encompassing a HeX-B can be about 1%, about 5%, about 10%, about 25%, about 50% higher than a parent protein under the same conditions. In still other aspects, methods of generating an engineered protein as disclosed herein may increase the T_{M} of the engineered protein about 0.5° C. to about 10° C. higher than a parent protein under the same conditions. In yet other aspects, methods of generating an engineered protein as disclosed herein may increase the T_{M} of an engineered protein disclosed herein encompassing a HeX-B can be about 1° C., about 2° C., about 3° C., about 4° C., about 5° C., about 6° C., about 7° C., about 8° C., about 9° C., or about 10° C. higher than a parent protein under the same conditions.

In other embodiments, thermal stability can be determined based on melting entropy. Methods of placing a halogenated amino acid in a protein pocket disclosed herein may increase the enthalpy of melting (ΔH_{M}) of an engineered protein compared to a parent protein. In some aspects, methods of generating an engineered protein as disclosed herein may increase the ΔH_{M} of an engineered protein by about 1% to about 50% higher than a parent protein under the same conditions. In other aspects methods of generating an engineered protein as disclosed herein may increase the ΔH_{M} of an engineered protein by about 1%, about 5%, about 10%, about 25%, about 50% higher than a parent protein under the same conditions. In some aspects the ΔH_{M} of an engineered protein disclosed herein encompassing a HeX-B can be about 1.0 kcal/mol can be more than 1 kcal/mol higher than a parent protein under the same conditions. In still other aspects, methods of generating an engineered protein as disclosed herein may increase the ΔH_{M} of an engineered protein by about 1.0 kcal/mol to about 20.0 kcal/mol higher than a parent protein under the same conditions. In other aspects, methods of generating an engineered protein as disclosed herein may increase the ΔH_{M} of an engineered protein by about 1.0 kcal/mol, 2.0 kcal/mol, about 5.0 kcal/mol, about 10.0 kcal/mol, about 15 kcal/mol, or about 20.0 kcal/mol. In yet other aspects, methods of generating an engineered protein as disclosed herein may increase the ΔH_{M} of an engineered protein by about 1.0 kcal/mol, about 1.5 kcal/mol, about 2.0 kcal/mol, about 2.5 kcal/mol, about 3.0 kcal/mol, about 3.5 kcal/mol, about 4.0 kcal/mol, about 4.5 kcal/mol, 5.0 kcal/mol, about 5.5 kcal/mol, 6.0 kcal/mol, about 6.5 kcal/mol, 7.0 kcal/mol, about 7.5 kcal/mol, or about 8.0 kcal/mol higher than a parent protein under the same conditions.

After synthesis/production of an engineered protein as described herein, the engineered protein can be purified. Methods of protein purification are known in the art. By way of non-limiting examples, in some embodiments proteins may be purified by chromatography, molecular filtration, gel filtration, immunoadhesion, tag-selection, or a combination thereof.

Engineered proteins as disclosed herein can also be crystallized. Methods of protein crystallized are known in the art. By way of non-limiting examples, in some embodiments proteins may be crystallized by the bicelle method, the lipidic cubic phase method, the hanging drop vapor diffusion method, or a combination thereof.

(c) Methods of Determining the Properties of Engineered Proteins.

The various properties of an engineered proteins disclosed herein can be determine using a variety of known methods. In some aspects, properties of an engineered proteins disclosed herein may be assessed by x-ray data collection and structure determination, differential scanning calorimetry (DSC), quantum mechanical (QM) calculations, turbidity assays, to a combination thereof.

(i) x-Ray Data Collection and Structure Determination

In various embodiments, properties of an engineered proteins disclosed herein may be assessed by x-ray data collection and structure determination. Methods of protein crystallization are known in the art. By way of non-limiting example, crystals of engineered proteins disclosed herein can be subjected to cryogenic nitrogen stream on an Advanced Light Source (ALS) Beamline. In some aspects, the resulting diffraction data from the ALS beamline can be reduced using a commercially available software package such as, by means of non-limiting example, XDS and CCP4 suite.

In various aspects, X-ray data can be phased by molecular replacement by applying the atomic coordinates of the parent protein from the Protein Data Bank (PDB) as a starting model. This method can yield initial models with R_{work} values and R_{free} values. In some aspects, X-ray data can be used to refine the engineered protein structure using crystallographic software.

Non-limiting examples of parameters that can be assessed by x-ray data collection and structure determination include resolution (Δ), total reflections, unique reflection, multiplicity, completeness, mean l/σ, R_{merge}, Rₘₑₐₛ, R_{work}, R_{free}, non-solvent atoms, solvent atoms and average b-factor.

(ii) Differential Scanning Calorimetry (DSC)

In various embodiments, properties of an engineered proteins disclosed herein may be assessed by DSC. Methods of DSC are known in the art. In some aspects, engineered proteins can be subjected to multiple melting cycles to determine melting curves of the engineered proteins. By way of non-limiting example, engineered proteins can be subjected to heating cycles ranging from about 10° C. to 90° C. at a scan rate of about 0.5, 0.75, or 1.0° C./min. In some aspects, engineered proteins can be subjected to cooling scans and subsequent heating cycles to determine reversibility. By way of non-limiting example, engineered proteins can be subjected to cooling cycles ranging from about 90° C. to 10° C. at a scan rate of about 0.25, 0.5, or 0.75° C./min.

In various embodiments, melting and reversibility data can be analyzed to determine thermodynamic parameters of engineered proteins disclosed herein. Non-limiting examples of thermodynamic parameters that can be obtained by DSC include the specific heat capacities (ΔCₚ), melting temperatures (T_{M}), melting enthalpies (ΔH_{M}) and ΔH_{fit}/ΔH_{cal} ratios.

(iii) Quantum Mechanical (QM) Calculations

In various embodiments, properties of an engineered proteins disclosed herein may be assessed by quantum mechanical (QM) calculations. Methods of QM calculation are known in the art. By means of non-limiting example, QM energies and electrostatic potential maps (ESPs) of engineered proteins disclosed herein can be calculated using a Gaussian 09 revision E.01 with a Møller-Plesset second order (MP2) in a solvent. This low-dielectric solvent model can be appropriate for calculations on systems that involve explicit solvent and short distances between interacting atoms and reflects the low dielectric expected for a protein interior.

In some aspects, QM calculations can validate geometries and energies in model DNA junction systems. In some aspects, QM calculations can determine the atomic coordinates of the interacting residues in engineered proteins disclosed herein. In still other aspects, QM calculations can determine if hydrogen atom positions were geometry-optimized in engineered proteins disclosed herein with a semiempirical AM1 calculation. In yet other aspects, QM calculations can determine the torsional angle, δ, of a hydroxyl hydrogen in an engineered protein disclosed herein to assess if manually rotation can contribute to a HeX-B.

(iv) Activity

In various embodiments, properties of an engineered proteins disclosed herein may be assessed by determining its level of activity as compared to the activity of the parent protein under similar conditions. By way of a non-limiting example, the activity of an engineered protein that is an enzyme may be compared to the activity of the parent enzyme.

### III. Use of Engineered Proteins Including at Least One Halogenated Amino Acid Residue

As will be appreciated by those of skill in the art, the presently disclosed engineered protein can be used in a wide variety of applications. By way of non-limiting examples they made be used for research, industrial, manufacturing, therapeutic, diagnostic and/or biotechnological applications. The increased stability of the disclosed engineered protein provides numerous advantages for these applications. For example, engineered proteins as disclosed herein may have extended lifetimes or storage at ordinary or increased temperatures, may result in higher yields of soluble protein during manufacture, may be used in artificial environments, and/or may be more ‘evolvable’ or able to acquire beneficial traits for a given environment.

Engineered proteins disclosed herein may be used as a therapeutic protein drug. The use of engineered proteins as disclosed herein as therapeutic proteins may have a wide variety advantages over parent proteins. For example, in a non-limiting example, when used as a therapeutic protein drug, an engineered therapeutic protein may have an increased circulating half-life compared to the parent protein under the same conditions. By way of another non-limiting example, engineered therapeutic proteins as disclosed herein may exhibit an increased ability to bind to a target as compared to the parent protein under the same conditions. Additionally, engineered proteins disclosed herein when used as a therapeutic protein may have a longer storage lifetime as compared to the parent protein under the same conditions. By way of a further example, engineered proteins disclosed herein when used as a therapeutic protein may have increased stability at higher temperatures compared to the parent protein.

Engineered proteins disclosed herein may be used in manufacturing applications. Non-limiting examples of manufacturing applications that engineered proteins disclosed herein may be used in include food, pharmaceutical, and textile manufacturing. The use of engineered proteins as disclosed herein in manufacturing applications may have numerous advantages over parent proteins. By way of a non-limiting example, engineered proteins disclosed herein may be produced in larger scale than parent proteins. In some aspects, synthesis/production of engineered proteins may have a 2-fold to 10-fold increase in scaled synthesis/production compared to parent proteins. In another non-limiting example, engineered proteins disclosed herein can be used for the production of other proteins in higher quantities than when parent proteins are used. In a further example, engineered proteins disclosed herein can be used for synthesis/production of other proteins at temperatures higher than would be permissible using parent proteins.

Engineered proteins disclosed herein may be used for research applications. Non-limiting examples of research applications that engineered proteins disclosed herein may be used in include proteome research, structural characterization of proteins, genomic research, and metabolic research. The use of engineered proteins as disclosed herein may have a wide variety advantages over parent proteins in research applications. For example, engineered proteins disclosed herein may decrease cost and/or time of completion of research applications than when parent proteins are used. By way of another non-limiting example, engineered proteins as described herein for use as research agents, may have an increased shelf life as compared to a parent protein.

Engineered proteins disclosed herein may also be used in industrial applications. Non-limiting examples of industrial applications that engineered proteins disclosed herein may be used in include fermentation-based food production, textile processing, leather processing, and cellulosic ethanol production. The use of engineered proteins as disclosed herein may have a wide variety advantages over parent proteins in industrial applications. By way of a non-limiting example, engineered proteins disclosed herein may decrease energy requirements of industrial applications as compared to parent proteins.

Engineered proteins disclosed herein may be used in diagnostic applications. Non-limiting examples of diagnostic applications that engineered proteins disclosed herein may be used in include molecular diagnostics, diagnostic imaging, environmental diagnostics, and agricultural diagnostics. The use of engineered proteins as disclosed for diagnostic applications may have numerous advantages over parent proteins. By way of non-limiting examples, engineered proteins disclosed herein may have a higher rate of detection than parent proteins, and/or may be used and stored at higher temperatures than parent proteins.

Engineered proteins disclosed herein may be used in biotechnological applications. Non-limiting examples of biotechnological applications that engineered proteins disclosed herein may be used in include bioreactors, enzyme electrodes, and biocatalysts. The use of engineered proteins as disclosed for biotechnological applications may have multiple advantages over parent proteins. For example, engineered proteins disclosed herein can have a higher rate of biosynthesis than when parent proteins are used.

### EXAMPLES

The following examples are included to demonstrate various embodiments of the present disclosure. It should be appreciated by those of skill in the art that the techniques disclosed in the examples that follow represent techniques discovered by the inventors to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Introduction to Examples 1-5

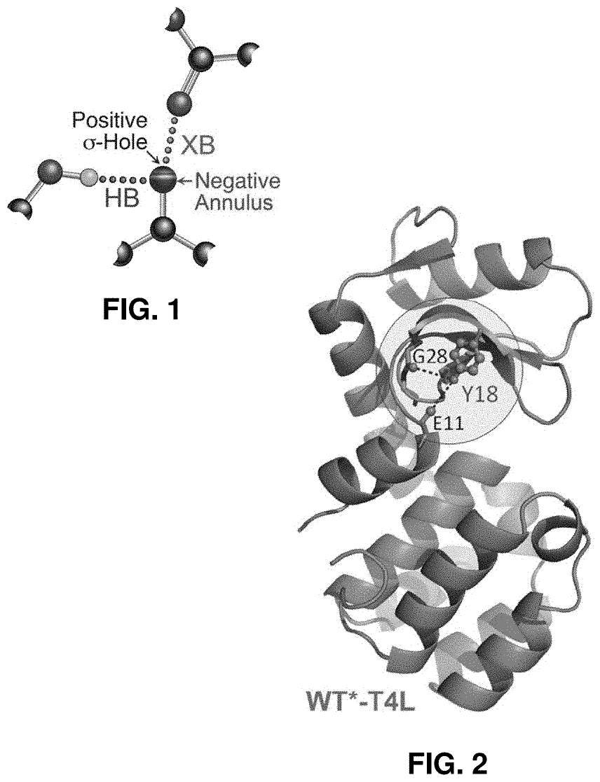

XBs are analogous to HBs and, although the physicochemical foundation remains debated, the electrostatic nature of XBs is readily modeled by the σ-hole theory (FIG. 1). In the model of FIG. 1, one lobe of a halogen's p_{z} orbital becomes depopulated when its valence electron is subsumed by the molecular σ-orbital of a covalent bond. The result is an electropositive σ-hole, which serves as the XB donor. However, the electronegative annulus around the waist makes the halogen amphoteric, able to serve simultaneously as an HB acceptor perpendicular to the σ-hole.

In biology, XBs define the binding specificity and affinity of halogenated enzyme inhibitors and have been shown to affect the folding of nucleic acids, making them important tools for both medicinal chemistry and biomolecular engineering. To study the effects of XBs on protein folding and enhanced σ-hole on the XB donor ability, a lysozyme from the T4 bacteriophage (T4L) was used as a model system for the following studies. T4L has long served as a model system for protein thermodynamics and stability by multiple researchers. There are now >300 studies of T4L point mutations that demonstrate the power of this system to study structure-energy-function relationships in a well-controlled protein, and it is the rare instance that an engineered mutation, including those with halogenated amino acids, results in significant stabilization (≥1° C. increase in the melting temperature, T_{M}) of the enzyme.

### Example 1

A series of T4L constructs were designed in which Y18 is halogenated at the meta position (^{mX}Y18-T4L, where X═Cl, Br, or I), leaving the critical OH substituent intact. The halogens are placed to potentially form an XB to the carbonyl oxygen of G28 located in a tight loop region near the enzyme active site and thus enhance T4L stability. The G28 oxygen forms a standard (3.1 Å) HB to the backbone amino nitrogen of arginine 14 [R14 (FIG. 2)]. However, a small void space was identified within this loop region that could potentially accommodate a halogen to form an XB to this G28 oxygen in a geometry that is perpendicular to the O ⋅ ⋅ ⋅ H—N HB.

The crystal structures of the ^{mX}Y18-T4Ls (^{mCl}Y18-T4L, ^{mBr}Y18-T4L, and ^{mI}Y18-T4L) were determined from 1.35 Å to 1.65 Å resolution under the crystallographic parameters in Table 1.

TABLE 1

| | | | |
|---|---|---|---|
| Parameter | ^{mCl}Y18-T4L | ^{mBr}Y18-T4L | ^{ml}Y18-T4L |

Crystal

| | | | |
|---|---|---|---|
| Unit Cell Lengths | a = b = 59.61 | a = b = 60.43 | a = b = 60.53 |
| | c = 95.12 | c = 96.66 | c = 96.35 |
| Space Group | P3₂21 | P3₂21 | P3₂21 |

Data Collection Statistics

| | | | |
|---|---|---|---|
| Resolution (Å)¹ | 34.68-1.36 (1.41-1.36) | 32.22-1.35 (1.40-1.35) | 35.47-1.65 (1.71-1.65) |
| # Total Reflections¹ | 561,537 (38,239) | 571,355 (40,977) | 251,506 (25,268) |
| # Unique Reflections¹ | 42,545 (4,168) | 45,223 (4,450) | 25,201 (2,466) |
| Multiplicity | 13.2 | 12.6 | 10.0 |
| Completeness¹ | 99% (99%) | 99% (99%) | 99% (99%) |
| Mean I/σ (I)¹ | 23.87 (1.97) | 19.55 (1.81) | 15.60 (1.51) |
| R_{merge}¹ | 0.069 (1.191) | 0.069 (1.269) | 0.088 (1.303) |
| Rₘₑₐₛ¹ | 0.071 (1.263) | 0.071 (1.347) | 0.093 (1.372) |
| CC_{1/2} | 1.00 (0.72) | 1.00 (0.73) | 1.00 (0.71) |

Structure Refinement Statistics
Final Model Statistics

| | | | |
|---|---|---|---|
| R_{work}¹ | 0.177 (0.321) | 0.186 (0.395) | 0.199 (0.296) |
| R_{free}¹ | 0.208 (0.309) | 0.216 (0.412) | 0.232 (0.330) |
| Non-Solvent Atoms | 1,600 | 1,514 | 1,535 |
| Solvent Atoms | 383 | 373 | 241 |
| Average B-factor | 15.74 | 20.80 | 28.10 |
| PDB Code | 5V7E | 5V7D | 5V7F |

¹Values for the highest resolution shell are shown in parentheses.

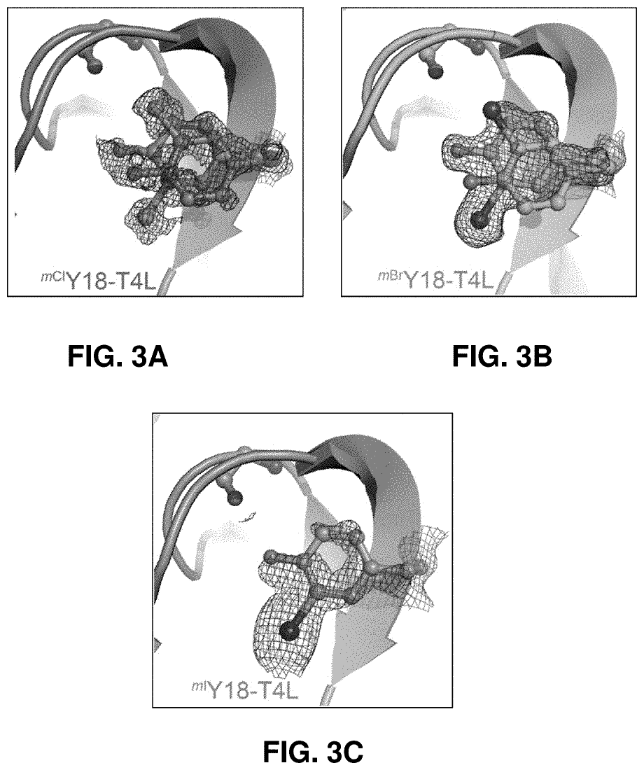

Fo−Fc difference maps (at 1.2a level), known also as a omit electron density map, were used to show how well the structural model fits the experimentally collected data for ^{mCl}Y18-T4L (FIG. 3A), ^{mBr}Y18-T4L (FIG. 3B), and ^{mI}Y18-T4L (FIG. 3C).

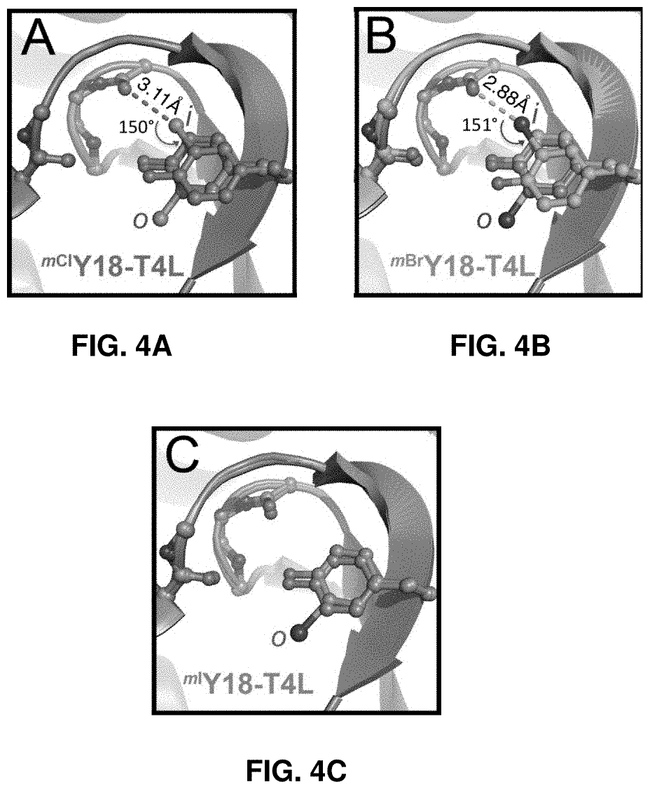

The crystal structures of ^{mCl}Y18-T4L (FIG. 4A), ^{mBr}Y18-T4L (FIG. 4B), and ^{mI}Y18-T4L (FIG. 4C) were all isomorphous with the parent WT* (FIG. 2), allowing any observed structural perturbations to be analyzed relative to each other, and relative to their effects on the constructs' stabilities and activities. The ^{mX}Y18 side chain of each construct sat in a pocket formed by a pair of antiparallel loops and was rotated to place the halogen inside this pocket (i-rotamer) pointed toward G28 or outside the pocket (o-rotamer) exposed to the solvent (FIGS. 4A-4C). The i-rotamer propensity increases as the halogen becomes smaller (Table 2). The percent relative to maximum exposure (% max) was calculated relative to the exposure of each halogen in an isolated ^{mX}Y amino acid residue (SAS for Cl of ^{mCl}Y=124.8 Å², SAS for Br of ^{mBr}Y=132.7 Å², and SAS for I of ^{mI}Y=143.6 Å²) (Table 2).

TABLE 2

| | i- | | | |
|---|---|---|---|---|
| | rotamer:o- | R_{x•••o} | | |
| Construct | rotamer | (%ΣR_{vdw})^{a} | θ₁^{b} | SAS (% max)^{c} |
| ^{mCl}Y18-T4L | 54:46 | 3.11 Å (95%) | 150° | 17.3 Å² (13.8%) |
| ^{mBr}Y18-T4L | 22:78 | 2.88 Å (85%) | 151° | 28.9 Å² (21.8%) |
| ^{ml}Y18-T4L | 0:100 | not applicable | not | 17.1 Å² (11.9%) |
| | | | applicable | |

Where ^{a}is %ΣR_{vdw} is the percent of the sum of the standard van der Waals radii of the halogen to oxygen (X•••O) interacting pair;
^{b}is θ₁ is the angle of approach of the oxygen acceptor to the halogen, ∠ (C—X•••O); and
^{c}is solvent accessible surfaces for halogen atoms were calculated using PyMol.

The structure of the loop is invariant among all constructs, with the O ⋅ ⋅ ⋅ H—N HB distance (from G28 to R14) varying by <0.1 Å relative to WT* (Tables 3-6). This loop region in the crystal structure was, therefore, very rigid, sterically constraining the size of the halogen that was accommodated and consequently its potential to participate in an XB.

TABLE 3
Residue•Residue Interactions

| Residue (substituent) | E11 (O═C_{b}) | G28 (O═C_{b}) |
|---|---|---|
| Y18 (OHₛ) | 4.1 Å | 4.1 Å |
| R14 (N_{b}) | — | 3.1 Å |

Residue•Water Interactions

| | W1 | W2 | W3 | W4 | W5 | W6 |
|---|---|---|---|---|---|---|
| Y18 (OHₛ) | 3.1 Å | 3.8 Å | — | 3.0 Å | — | — |
| E11 (O═C_{b}) | 2.8 Å | — | 3.0 Å | — | — | — |
| E11 (O═Cₛ) | — | — | — | 2.9 Å | — | — |

Water•Water Interactions

| | W2 | W3 | W4 | W5 | W6 |
|---|---|---|---|---|---|
| W1 | 2.7 Å | — | — | — | — |
| W2 | — | 2.2 Å | — | — | — |
| W3 | — | — | 2.6 Å | — | — |
| W4 | — | — | — | 2.8 Å | — |
| W5 | — | — | — | — | 3.1 Å |

Interacting atoms are highlighted in bold. Groups with a subscript “b” indicate backbone atoms, while those with subscript “s” are side chain atoms.

TABLE 4
Residue•Residue Interactions^{1,2}

| Residue (substituent) | E11 (O═C_{b}) | G28 (O═C_{b}) |
|---|---|---|
| Y18 (OHₛ-i) | 3.9 Å | 4.6 Å |
| Y18 (OHₛ-o) | 4.1 Å | 3.7 Å |
| Y18 (Clₛ-i) | | 3.1 Å |
| R14 (N_{b}) | | 3.1 Å |

Residue•Water Interactions^{1,2,3}

| Y18 (OHₛ-i) | W1-i | W1-o | W2 | W3-i | W3-o | W4 | W5 | W6 |
|---|---|---|---|---|---|---|---|---|
| Y18 (OHₛ-o) | 2.6 Å | (3.0 Å) | | | | — | — | — |
| Y18 (OHₛ-o) | (2.0 Å) | 3.0 Å | | | | — | — | — |
| Y18 (Clₛ-i) | 3.2 Å | (3.4 Å) | | | | — | — | — |
| Y18 (Clₛ-o) | — | — | — | 3.6 Å | 2.7 Å | 2.7 Å | 3.3 Å | 3.4 Å |
| E11 (O═C_{b}) | 2.7 Å | 2.8 Å | 3.2 Å | 2.9 Å | 3.0 Å | — | — | — |
| E11 (O═Cₛ) | — | — | — | 4.7 Å | 3.1 Å | 3.1 Å | — | — |

Water•Water Interactions^{2,3}

| | W1-o | W2 | W3-i | W3-o | W4 | W5 | W6 |
|---|---|---|---|---|---|---|---|
| W1-i | (1.8 Å) | 3.7 Å | — | — | — | — | — |
| W1-o | — | 2.6 Å | — | — | — | — | — |
| W2 | — | — | 2.4 Å | 3.7 Å | — | — | — |
| W3-i | — | — | — | (2.0 Å) | — | — | — |
| W3-o | — | — | (2.0 Å) | — | 4.0 Å | 2.9 Å | — |
| W4 | — | — | — | — | — | 2.9 Å | — |
| W5 | — | — | — | — | — | — | 2.3 Å |

Where
¹is interacting atoms are highlighted in bold; groups with a subscript “b” indicate backbone atoms, while those with subscript “s” are side chain atoms;
²is rotamer with the halogen placed inside the loop is designated “-i” and outside as “-o”; and,
³is numbers in parentheses are for inside vs outside to indicate how these were assigned to the two rotamers.

TABLE 5
Residue•Residue Interactions^{1,2}

| Residue (substituent) | E11 (O═C_{b}) | G28 (O═C_{b}) |
|---|---|---|
| Y18 (OHₛ-i) | 4.3 Å | 4.8 Å |
| Y18 (OHₛ-o) | 4.4 Å | 3.5 Å |
| Y18 (Brₛ-i) | | 2.9 Å |
| R14 (N_{b}) | | 3.1 Å |

Residue•Water Interactions^{1,2,3}

| Y18 (OHₛ-i) | W1-i | W1-o | W2 | W3 | W4-i | W4-o | W5-i | W5-o | W6 |
|---|---|---|---|---|---|---|---|---|---|
| Y18 (OHₛ-o) | 2.7 Å | (3.3 Å) | — | 2.6 Å | 2.4 Å | (2.6 Å) | — | — | — |
| Y18 (OHₛ-o) | (2.0 Å) | 3.0 Å | — | 2.8 Å | (3.2 Å) | 2.8 Å | — | — | — |
| Y18 (Brₛ-i) | 3.3 Å | 3.3 Å | 2.9 Å | — | | — | — | — | — |
| Y18 (Brₛ-o) | — | — | 3.9 Å | 3.3 Å | 2.2 Å | 3.3 Å | 3.5 Å | 2.5 Å | — |
| E11 (O═C_{b}) | 2.7 Å | 2.8 Å | 3.3 Å | 3.1 Å | 3.7 Å | 2.6 Å | — | — | — |
| E11 (O═Cₛ) | — | — | — | — | 3.4 Å | 2.8 Å | — | — | 3.1 Å |

Water•Water Interactions^{2,3}

| | W1-o | W2 | W3 | W4-i | W4-o | W5-i | W5-o | W6 |
|---|---|---|---|---|---|---|---|---|
| W1-i | (1.8 Å) | 3.5 Å | — | — | — | — | — | — |
| W1-o | — | 2.7 Å | — | — | — | — | — | — |
| W2 | — | — | 2.6 Å | | — | — | — | — |
| W3 | — | — | — | 2.2 Å | 2.7 Å | — | — | — |
| W4-i | — | — | — | — | (1.5 Å) | 3.8 Å | — | 2.9 Å |
| W4-o | — | — | — | — | — | (2.3 Å) | — | 3.3 Å |
| W5-i | — | — | — | 3.0 Å | — | — | (1.2 Å) | 3.1 Å |
| W5-o | — | — | — | | — | — | — | 2.8 Å |

Where
¹is interacting atoms are highlighted in bold; groups with a subscript “b” indicate backbone atoms, while those with subscript “s” are side chain atoms;
²is rotamer with the halogen placed inside the loop is designated “-i” and outside as “-o”; and,
³is numbers in parentheses are for inside vs outside to indicate how these were assigned to the two rotamers.

TABLE 6
Residue•Residue Interactions

| Residue (substituent) | E11 (O═C_{b}) | G28 (O═C_{b}) |
|---|---|---|
| Y18 (OHₛ) | 4.6 Å | 2.9 Å |
| R14 (N_{b}) | — | 3.2 Å |

Residue•Water Interactions

| | W2 | W3 | W4 | W5 | W6 | W7 | W8 |
|---|---|---|---|---|---|---|---|
| Y18 (OHₛ) | 4.0 Å | — | — | — | — | — | — |
| Y18 (I) | — | — | 3.1 Å | — | 2.3 Å | 4.1 Å | 3.8 Å |
| E11 (O═C_{b}) | 3.1 Å | — | 2.5 Å | — | — | — | — |
| E11 (O═Cₛ) | — | 2.6 Å | — | — | — | — | — |

Water•Water Interactions

| | W3 | W4 | W5 | W6 | W7 | W8 |
|---|---|---|---|---|---|---|
| W2 | 3.0 Å | 3.6 Å | — | — | — | — |
| W3 | — | — | — | — | — | — |
| W4 | — | — | — | — | — | — |
| W5 | — | — | — | 1.8 Å | — | — |
| W5 | — | — | — | — | 3.2 Å | — |
| W5 | — | — | — | — | — | 2.6 Å |

Interacting atoms are highlighted in bold. Groups with a subscript “b” indicate backbone atoms, while those with subscript “s” are side chain atoms.

The halogens of the i-rotamers of ^{mCl}Y18-T4L and ^{mBr}Y18-T4L were seen to form short-range interactions with the carbonyl oxygen of G28 (FIGS. 4A-4B). The Cl ⋅ ⋅ ⋅ O distance in ^{mCl}Y18-T4L was ˜95% of the sum of the standard van der Waals radii (ΣR_{vdw}) of the interacting atoms, near the optimum distance for biological XBs, while the Br ⋅ ⋅ ⋅ O distance in ^{mBr}Y18-T4L was much shorter at ˜85%. The angles of approach of the oxygen acceptor to the halogen (θ₁=150° for O ⋅ ⋅ ⋅ Cl—C, and θ₁=151° for O ⋅ ⋅ ⋅ Br—C) were shallow relative to the ideal linear approach (θ₁=180°); however, these geometries were well within the range of XB interactions observed in biological systems and, as will be discussed later, were accommodated by additional polarization of the halogens in this particular system. In addition, the approach angles of halogen to the acceptor HB (X ⋅ ⋅ ⋅ O ⋅ ⋅ ⋅ N) were 80.6° for X═Cl and 82.2° for X═Br, which are consistent with the XBs being an orthogonal interaction (geometrically perpendicular and energetically independent) to the HB. Thus, these interactions were classified as XBs.

The small displacement of the ^{mCl}Y18-T4L aromatic side chain in the i-rotamer from the o-rotamer position was likely an attempt to pull the halogen into a more linear XB geometry. The larger displacement of the ^{mBr}Y18-T4L side chain away from G28, however, suggested destabilizing steric effects in the i-rotamer even as the halogen forms a short XB interaction. This balance between an XB attractive and steric repulsive force (and potentially bonding forces from distortion of the side chain) would account for the lower i-rotamer:o-rotamer ratio of the bromo construct.

### Example 2

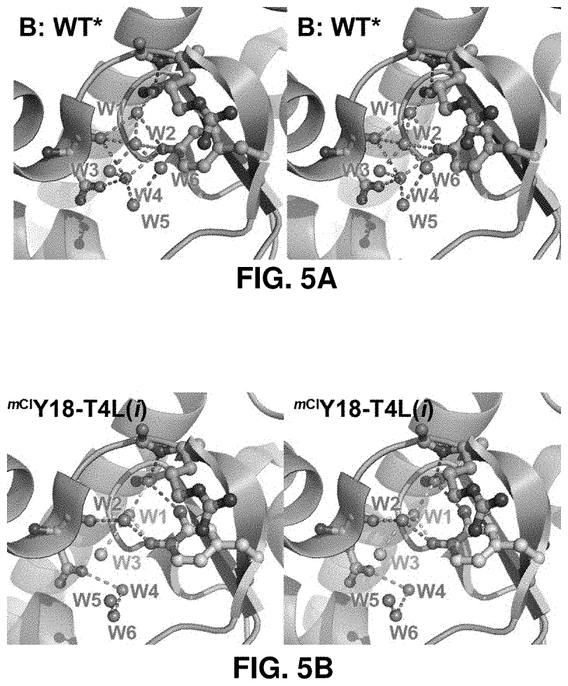

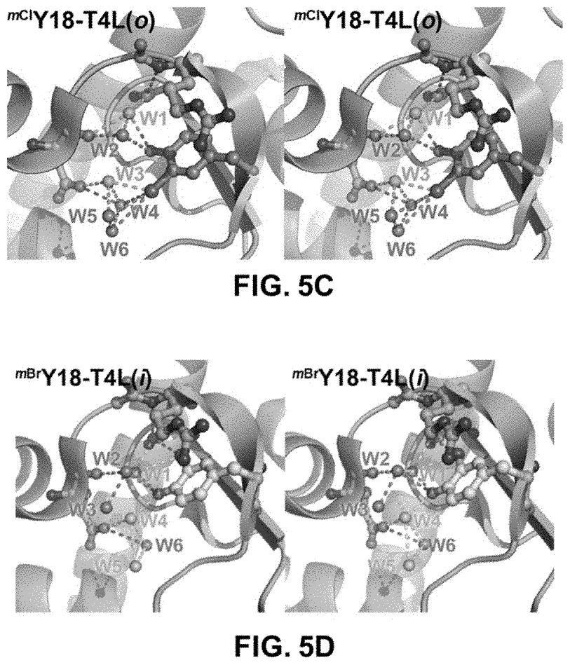

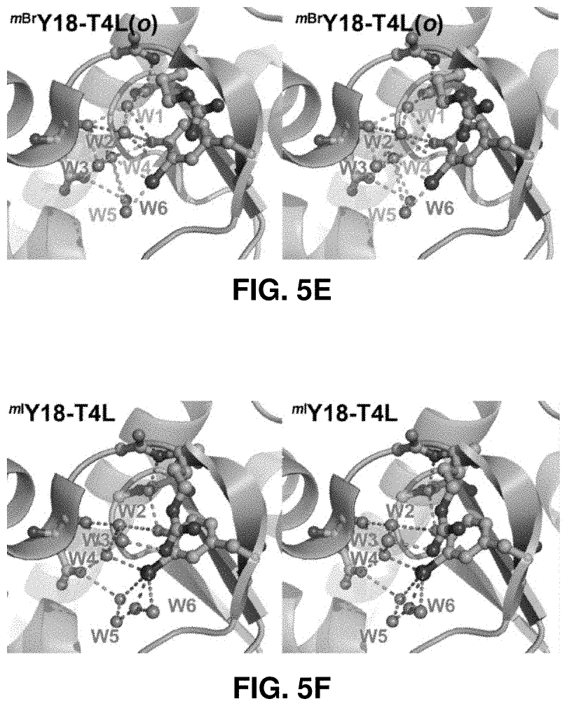

To determine how XBs affect protein solvent structure, the constellation of waters around E11, Y18, and G28 seen in the WT* structure (FIG. 5A) was mapped to those residues within the halogenated constructs ^{mCl}Y18-T4L (FIGS. 5B-C), ^{mBr}Y18-T4L FIGS. 5D-E), and ^{mI}Y18-T4L (FIG. 5F). The constellation of waters around E11, Y18, and G28 seen in the WT* structure remained mostly intact in the halogenated constructs, except to accommodate the halogens in their i- or o-rotamers (FIGS. 5B-F and Tables 3-6). For ^{mCl}Y18-T4L, the waters that bridge Y18 to E11 (W2-W6) were seen in positions similar to those in WT*, with the exceptions of W1 and W3 (FIGS. 5B-5C). In the chlorinated construct, the position of W1, which is particularly important in stabilizing the T4L protein, was filled by two partially occupied water molecules, each very close (within 1.8 Å) to the other. In addition, one of these waters sat unusually close to the OH of the Y18 side chain of the o-rotamer. This this water was interpreted as being a single molecule occupying two mutually exclusive positions: one assigned to the o-rotamer (W1-o, sitting in nearly the same position as W1 in WT*) and the other to the i-rotamer (W1-i, repositioned to sit in the aromatic plane) of the ^{mCl}Y18 residue. Although not as important as W1 in terms of defining protein stability, W3 also showed two partially occupied positions, one that forms an HB to the Cl of the o-rotamer (assigned as W3-o) and one that does not (assigned as W3-i).

The waters around ^{mBr}Y18-T4L (FIGS. 5D-5E and Table 5) showed patterns similar to those in the ^{mCl}Y18-T4L structure, with certain solvent positions (including W1) occupied by molecules that were associated with the i-rotamer or the o-rotamer. The solvents in ^{mI}Y18-T4L (FIG. 5F and Table 6) are similar to WT*, except that W1 is entirely missing, a consequence of the Y18 side chain being pushed closer to the carbonyl oxygen of G28, which has either completely displaced this solvent molecule or made it less specific in its positions (thereby making it unobservable in the electron density map).

### Example 3

As observed in EXAMPLE 1, the fact that the larger iodine of ^{mI}Y18-T4L was entirely in the o-rotamer supported this interpretation. Given that none of the constructs were entirely in the i-rotamer position, the question was whether the XB interactions are actually stabilizing. This question was addressed by comparing the melting temperature (T_{M}) and enthalpy (ΔH_{M}) in solution of each ^{mX}Y18-T4L construct to those of the parent WT* enzyme. Specifically, differential scanning calorimetry (DSC) was used to determine how the conformational features seen in each of the crystal structures affect the stability of the protein. This protein system allows precise determination of melting temperatures and enthalpies and, thus, allows us to accurately assign thermodynamic values to molecular interactions associated with specific structural modifications.

The DSC-measured T_{M} of ^{mI}Y18-T4L (Table 7), with the iodine entirely in the exposed o-rotamer position, was ˜0.5° C. lower than that of WT*, which showed that a protein can be destabilized when a hydrophobic methyl or halogen substituent is added to a solvent-exposed position and that hydrophobic side chains effect T4L stability.

TABLE 7

| | | ΔH_{M} | ΔS_{M} | ΔCₚ (kcal |
|---|---|---|---|---|
| construct | T_{M} (° C.) | (kcal/mol) | (cal mol⁻¹ K⁻¹)^{a} | mol⁻¹ K⁻¹) |
| WT* | 57.30 ± 0.01 | 120.2 ± 0.5 | 363.8 ± 1.5 | 2.6 ± 0.2 |
| ^{mCl}Y18-T4L | 58.28 ± 0.01 | 122.9 ± 0.4 | 370.7 ± 1.2 | 2.9 ± 0.3 |
| ^{mBr}Y18-T4L | 57.36 ± 0.02 | 119.2 ± 0.4 | 360.6 ± 1.1 | 3.3 ± 0.2 |
| ^{ml}Y18-T4L | 56.78 ± 0.01 | 115.5 ± 0.6 | 350.1 ± 1.9 | 2.8 ± 0.1 |

^{a}ΔS_{M} is the melting entropy calculated as ΔH_{M}/T_{M}.

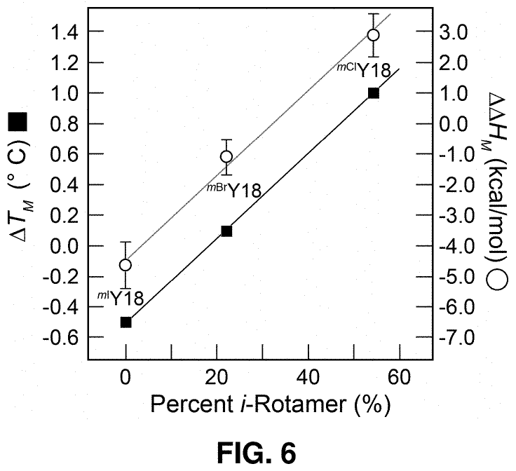

This hydrophobic effect was reflected in the increased ΔCₚ value. The ^{mBr}Y18-T4L construct had T_{M} and ΔH_{M} values that were very similar to that of WT*, indicating that the stabilizing XB in the i-rotamer nearly exactly counterbalanced the destabilizing effects of steric repulsion of this buried placement and the exposure in the o-rotamer. The most interesting case was that of the ^{mCl}Y18-T4L construct, which showed an ˜1° C. increase in T_{M} and a 2.7 kcal/mol increase in ΔH_{M} versus those of WT*. Together, the results showed that the increased stability of the protein, as measured by the T_{M} and ΔH_{M}, was dependent on the ability of the halogen to form an XB interaction in the i-rotamer (FIG. 6). Thus, for the first time, a more thermally stable protein was engineered by introducing a halogenated, in this case chlorinated, unnatural amino acid.

The entropy of melting (ΔS_{M}) can be calculated from the experimental ΔH_{M} and T_{M} values for each construct (Table 7). The resulting ΔS_{M} for ^{mCl}Y18-T4L is −7 cal mol⁻¹ K⁻¹ higher than that of WT*, suggesting that the XB makes the protein more conformationally rigid. The alternative interpretation would be that ΔS_{M} was defined by changes in the solvent structure, particularly because the halogens of the ^{mX}Y residues are hydrophobic. The expectation was that if the halogen was already exposed to solvent, as was the case for the o-rotamer of ^{mI}Y18-T4L and ^{mBr}Y18-T4L constructs, the entropic change upon melting would be smaller than if the halogen were more buried, as in the i-rotamer of ^{mCl}Y18-T4L. To determine whether solvent effects were the primary determinant of ΔS_{M}, the solvent accessible surfaces were calculated (SASs (Table 2)) of the halogens in the i-rotamer (when present) and o-rotamer conformations. The halogens in the i-rotamer of ^{mCl}Y18-T4L and ^{mBr}Y18-T4L were fully buried, as reflected in SASs of 0 Å². The o-rotamers of all the halogenated constructs showed some degree of exposure to solvent, with the Br of ^{mBr}Y18-T4L being most exposed and the I of ^{mI}Y18-T4L being the least, particularly in terms of the percentage relative to the exposure of an isolated halogenated tyrosine. Unlike other studies, effect of each halogen in an exposed versus buried site was internally controlled here. This observation was contrary to what was expected, but careful analyses of the structures show that the side chain of Arg14 is pulled within HB distance (3.1 Å) of the I, thus burying a significant portion of the halogen surface that was otherwise solvent-exposed in the other constructs. A comparison of the SAS and associated solvent free energies showed that they were not correlated to the ΔS_{M} values. The ΔS_{M}s, however, were well correlated with the occupancies of the i-rotamers (R=99.3%), indicating that the interactions of the halogens with the loop are the primary determinants of the entropic effects on the protein structure. The DSC melting energies, converted to ΔG° of stability at 40° C., followed exactly the trend for the T_{M}s, showing that the XB rendered ^{mCl}Y18-T4L overall more stable than WT* at high temperatures.

Extrapolation of the thermodynamic DSC thermodynamic parameters to the standard temperature (25° C.) indicated that the overall stabilities, as reflected in ΔG°, for all of the halogenated constructs were lower than that of WT* at this lower temperature (Table 8).

TABLE 8

| | 25° C. | | | 40° C. | | |
|---|---|---|---|---|---|---|
| | ΔH ° | ΔS ° (cal | ΔG ° | ΔH ° | ΔS ° (cal | ΔG ° |
| | (kcal/ | mol⁻¹ | (kcal/ | (kcal/ | mol⁻¹ | (kcal/ |
| construct | mol) | K⁻¹) | mol) | mol) | K⁻¹) | mol) |
| WT* | −34.8 | −91.7 | −7.43 | −74.5 | −222 | −5.07 |
| ^{mCl}Y18-T4L | −27.6 | −67.6 | −7.38 | −70.5 | −208 | −5.31 |
| ^{mBr}Y18-T4L | −12.5 | −20.8 | −6.27 | −61.9 | −183 | −4.73 |
| ^{mI}Y18-T4L | −26.9 | −67.8 | −6.71 | −68.7 | −205 | −4.66 |

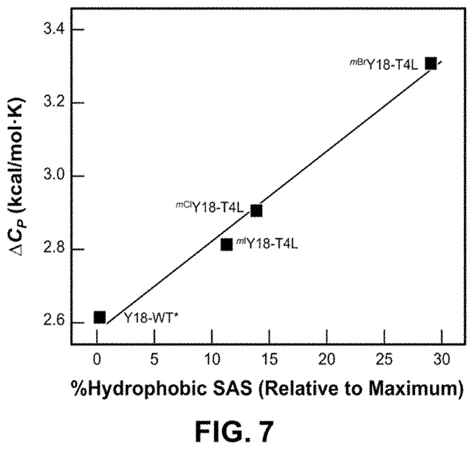

The resulting standard energies followed the general trend for the melting parameters previously reported for T4L, except for those of ^{mBr}Y18-T4L (ΔH° and ΔS°), which were calculated as being significantly lower than those of the other constructs. This singular outlier could be ascribed to the anomalously high ΔCₚ of ^{mBr}Y18-T4L, which affected extrapolation of its DSC energies to room temperature (RT). This higher DSC-measured ΔCₚ was indicative of a more hydrophobic system; ΔCₚ values have been shown to be well correlated with SASs. The experimental ΔCₚ values listed in Table 8 were indeed well correlated with the SAS values in Table 2, as the percent of the maximum exposure of the hydrophobic surface at Y18 (FIG. 7), indicated that they reflect features of the crystal structures. It was interpreted that the high ΔCₚ of ^{mBr}Y18-T4L may not be applicable to a RT calculation, because of temperature effects on the i-rotamer:o-rotamer ratio of the ^{mBr}Y18 side chain. An hypotheses was that the near the T_{M}, relaxation of the protein permitted the Br-substituted Tyr side chain to be better accommodated in the pocket and to form an XB, pushing a larger proportion to convert from the o- to i-rotamer. This allowed more exposure of nonpolar surfaces upon melting, resulting in the observed ΔCₚ near the T_{M} for ^{mBr}Y18-T4L being higher than would expected at RT. Indeed, extrapolation of the ^{mBr}Y18-T4L DSC data to RT using any of the other ΔCₚ values in Table 7 resulted in energies that were comparable to those of the other T4L constructs in this study. Similar effects on ΔCₚ would not be expected for either the Cl or I construct, because the side chain of ^{mI}Y18-T4L was not seen to form an XB, while the Cl of ^{mCl}Y18-T4L formed a stable XB. The XB favoring the buried i-rotamer of ^{mBr}Y18-T4L, however, was constricted by the steric repulsion that favors the exposed o-rotamer. In this way, the brominated construct exposed more hydrophobic surface upon thermal unfolding, which led to the anomalously high DSC-measured ΔCₚ value and the unexpectedly low ΔH° and ΔS° values from extrapolation to RT.

### Example 4

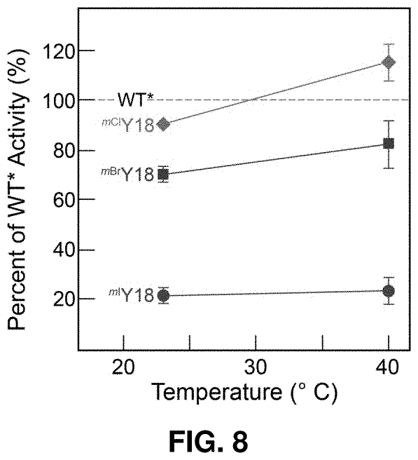

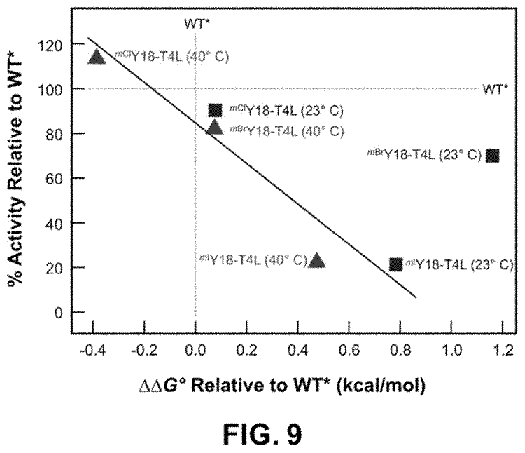

The thermal stabilities of T4L and its various mutants were correlated with the level of enzymatic activity. Thus, the standard bacterial clearing assay was used to monitor the effects of halogenation on the activity and, in addition, to provide additional support for their observed effects on the thermal stability of the enzyme. At RT (23° C.), the activities of the ^{mX}Y18-T4L constructs were all lower than that of WT* (FIG. 8) and, with the exception of that of ^{mBr}Y18-T4L (FIG. 9), were consistent with the ΔG° values calculated from the DSC melting energies (Table 8). At an elevated temperature (40° C.), the activity of the iodinated construct was not significantly changed but that of the brominated and chlorinated enzymes was increased relative to that of WT*, with ^{mCl}Y18-T4L becoming 15% greater than the native enzyme. The temperature at which ^{mCl}Y18-T4L would become more stable than WT* was predicted from extrapolation of the DSC values to be ˜35° C., which is also where one would expect the chlorinated enzyme to become more active. These general trends in activity at low and high temperatures followed and, therefore, confirmed the DSC melting results (with the singular exception of ^{mBr}Y18-T4L extrapolated to 25° C., as discussed above) and served to bridge the melting properties measured at high temperatures and the structural features of crystals grown at low temperatures. Thus, shown for the first time was that an XB can be specifically engineered not only to increase the thermal stability of a protein but also to increase its activity at elevated temperatures.

### Example 5

The thermal stabilities of each ^{mX}Y18-T4L construct, as reflected in the DSC-measured melting temperatures (T_{M}) and enthalpies (ΔH_{M}) (Table 7), were correlated with the percent i-rotamer (FIG. 6). The interactions (attributed here to XBs) within the loop conveyed stability to the ^{mX}Y18-T4L constructs, while exposure of the halogen to a solvent (similar to that previously seen with halogenated and methylated T4L analogues) destabilized the protein. These DSC values, however, may underestimate the stabilizing potential of the XB, particularly in the ^{mCl}Y18-T4L construct, which placed only 54% of the Cl in the i-rotamer position. If the Cl of ^{mCl}Y18-T4L was entirely in the XB position, the ΔH_{M} would be predicted to be 5.4 kcal/mol higher than that of WT*. In addition, the T_{M}s were well correlated with their ΔH_{M}s (a 1 kcal/mol increase in ΔH_{M} results in an ˜0.2° C. increase in T_{M}).

Even at ˜3 kcal/mol, the increase in ΔH_{M} measured for ^{mCl}Y18-T4L was significantly larger than that previously determined for Cl (0.5 kcal/mol) in a model DNA junction but comparable to those of Br and I (1.6-4.6 kcal/mol). The remarkably stronger Cl effect was attributed to an XB that was enhanced by an intramolecular HB from the hydroxyl substituent to the negative annulus of the halogen.

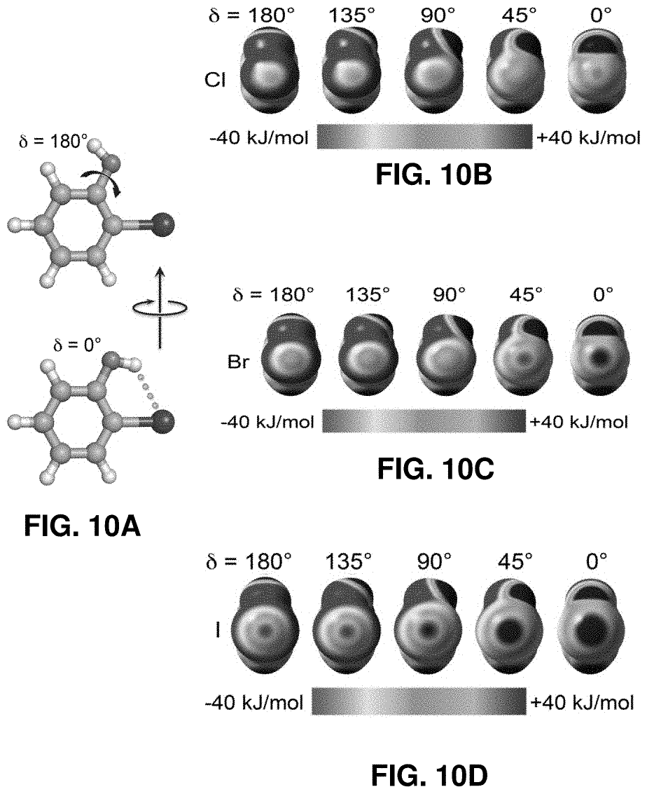

The electrostatic potential (ESP) was calculated as the OH is rotated from an angle δ of 180° (non-HB trans-OH orientation) to an angle δ of 0° (HB cis-OH orientation), in 45° increments (FIG. 10A). The QM-calculated ESPs were mapped onto the atomic surfaces of 2-halophenol (a model for the ^{mX}Y18 side chain), where the halogen is Cl (FIG. 10B), Br (FIG. 10C), or I (FIG. 10D). The ESP surface (FIGS. 10B-10D) showed that the σ-holes of halogen substituents became enhanced as the OH rotates from an angle δ of 180° (trans-OH) to an angle δ of 0° (cis-OH, pointing toward and within H-bonding distance of the halogen). This enhancement was interpreted as resulting from polarization of the electron density by the HB toward the p_{x,y} orbitals and away from the σ-hole, which renders the ESP for Cl comparable to that of the Br in bromobenzene.

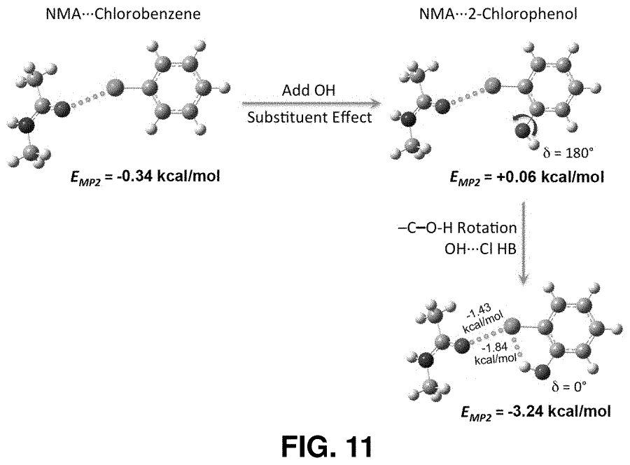

The effect of the enhanced σ-hole on the XB donor ability of the Cl can be appreciated by comparing the QM-calculated XB energies (E_{MP2}) for complexes of N-methylacetamide (NMA) with either chlorobenzene or 2-chlorophenol (a model for the XB complex between G28 and ^{mCl}Y18), positioned according to the ^{mCl}Y18-T4L crystal structure (FIG. 11). For chlorobenzene, the Cl ⋅ ⋅ ⋅ O XB energy was only slightly favorable (E_{MP2}=−0.3 kcal/mol), as expected for the inherently weak XB potential of Cl. The Cl was an even weaker XB donor (E_{X-MP2}=+0.06 kcal/mol) in the 2-chlorophenol complex with a trans-OH, reflecting the electron-donating property of the OH substituent, as suggested by calculations on ^{mI}Y-substituted insulin. The cis-OH, however, formed an HB with the Cl, resulting in an enhanced XB (E_{MP2}=−1.4 kcal/mol relative to the trans-OH). This E_{MP2} was nearly identical to that of an iodine XB (−1.8 kcal/mol) that was previously shown to rescue protein stability. Thus, the HB intensified the σ-hole and extended the allowed angles of approach by the acceptor to the halogen, both of which enhanced the XB potential of Cl, resulting in the HeX-B interaction. As the OH rotated from the trans-direction to the HeX-B cis-direction (FIG. 10B), the angle at which the calculated ESP switched from being a positive σ-hole to a negative annulus (a neutral-charge angle) was increased by 22-28° for the halogen substituent (from ˜160° to <135° for bromophenol, for example), allowing the relatively shallow 150° approach of the oxygen acceptor seen in the crystal structures to be stabilizing Cl and Br XB interactions. The HB to the halogen itself contributed significantly (−1.8 kcal/mol) to the interaction and, together with the HeX-B, accounted for the ˜3 kcal/mol enhancement of the DSC ΔH_{M} for ^{mCl}Y18-T4L versus that of WT*.

Why were these HeX-Bs not seen in all of the ^{mX}Y18-T4L constructs? For iodine, the answer was simply that this large halogen did not fit into the pocket of the rigid loop. The i-rotamer:o-rotamer ratio of the Br in ^{mBr}Y18-T4L reflected an ˜0.8 kcal/mol difference between a buried and exposed halogen. Although the Br ⋅ ⋅ ⋅ O geometry suggested a relatively strong XB, the large displacement of the side chain of the i-rotamer would indicate that this very short distance interaction was largely offset by an unfavorable steric clash. This balance between the opposing forces would account for the apparent discrepancy between the ΔG° and activity at RT (FIG. 9).

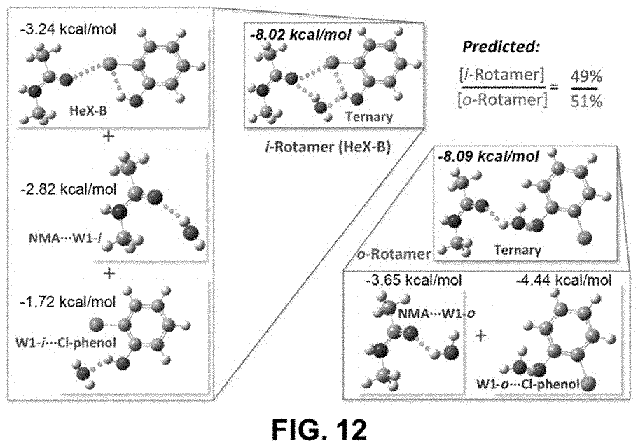

The answer to why only ˜50% of ^{mCl}Y18-T4L formed the HeX-B interaction comes again from considering the OH group, which had no sense for whether an XB was present. The Y18 hydroxyl was bridged by HBs to the carbonyl oxygen of E11 through a water (W1-i (FIGS. 4A-4C and FIG. 11)) which can form an HB to either the Cl (of the i-rotamer) or W1-o (the o-rotamer). The —OH ⋅ ⋅ ⋅ O═C distance between the Y18 hydroxyl and E11 carbonyl varied by <0.2 Å in ^{mCl}Y18-T4L (3.92 and 4.10 Å for the i- and o-rotamer, respectively, compared to 4.07 Å for WT*); consequently, the direct effects of the Cl on this interaction were expected to be minimal. If the OH of ^{mCl}Y18 was a donor to W1, it cannot simultaneously form an HB to the halogen and, thus, cannot enhance the XB capability; the approximate 1:1 i-rotamer:o-rotamer ratio suggested no preference for either Cl or W1. The higher energy of interaction of W1 with the ^{mCl}Y18 hydroxyl was a result of it being closer when this HB donor was not oriented toward the halogen. The QM energy calculated for a ternary complex of 2-chlorophenol, NMA, and W1 in their crystal structure conformations showed a <0.1 kcal/mol difference in E_{MP2} between the i- and o-rotamers, which would account for the near equal distribution among the rotamers (FIG. 12). Furthermore, 2-halophenols can adopt a trans-OH in water but a cis-OH with a weak intramolecular HB between the hydroxyl and halogen in organic solvents. The pocket where ^{mX}Y18 sits was partially solvent exposed, resulting in the hydroxyl having only a slight preference as an HB donor to the Cl over W1.

### Discussion of Examples 1-5

In this study, the question of whether addition of an XB to augment a critical HB by introducing an unnatural amino acid into the structure, would result in a more stable protein was addressed. The chlorinated ^{mCl}Y18-T4L construct demonstrated the potential application of XBs in increasing the stability and associated activity of an enzyme at elevated temperatures. As previously shown, halogenation of proteins generally has the effect of destabilizing protein structure, if the halogen is exposed to solvent and, therefore, incapable of forming an XB. This effect was recapitulated here, where the stabilities of the ^{mX}Y18-T4Ls were dependent on burying each halogen in a protein pocket (as reflected in the o- vs i-rotamers). The earlier study also showed that this destabilizing effect can be partially rescued if the halogen, particularly iodine with its very large σ-hole, can form an XB. Again, the same effect was observed in the current study; however, in this case, it was the chlorinated construct that formed the more stabilizing interaction, and the interaction was sufficiently strong not only to rescue the stability but also to increase it above that of WT*.

Although the ˜3 kcal/mol stabilization of T4L may seem to be small, it should be noted that proteins are stabilized by the concerted contributions of multiple low-energy, noncovalent interactions. Indeed, introducing a 3 kcal/mol interaction would convert metastable peptides and proteins to be fully stable structures, which would affect their functions. Similarly, adding a 3 kcal/mol interaction to a ligand to its interaction with a protein target would reduce its dissociation constant by >2 orders of magnitude, which in turn could make such a ligand more attractive as a potential drug candidate.

The stabilizing potential of an XB with a Cl donor had previously been determined to be very small (˜0.5 kcal/mol) in a DNA system. A Cl-XB had also been estimated from calculations to contribute as much as 1.5 kcal/mol to stabilizing the β-hairpin conformation of a cyclic peptide, one of the first demonstrations that an XB can potentially stabilize a protein-type conformation. Finally, it had been shown that addition of halogens that fill only void spaces contributes <0.8 kcal/mol per halogen atom. Thus, the ˜3 kcal/mol stabilization seen here with the addition of a single chlorine atom is surprising, leading to the question of why the Cl-XB has such as strong effect even compared to the previous I-XB in this same T4L protein system.

The improved ability of the Cl to serve as an XB donor attributes to an HB-enhanced XB. The HeX-B represents a new and potentially powerful variation on the stand-alone XB, expanding the standard menu of noncovalent interactions that dictate molecular folding. Because XBs and HBs share a common set of acceptors, their relationships can be complex. The interaction described herein, however, differed significantly from the orthogonal HB/XB interaction described previously in that the HB of the HeX-B is to the XB donor (as opposed to the acceptor) and enhanced, rather than being energetically independent of, the stabilizing potential of the interaction. Upon addition of an HB donor (including OH, SH, or NH₂) next to a halogen, it is now possible to enhance its XB potential through a synergistic relationship, beyond tuning through standard inductive effects. This enhancement is expected to be even more dramatic (˜3-4 kcal/mol) for anionic oxygen XB acceptors compared to the neutral carbonyl acceptor in our study (Tables 9 and 10) extending the range of stabilization potentials to as much as 6.7 kcal/mol for an iodine HeX-B (compared to the 6 kcal/mol for very strong HBs in proteins) when placed in an unconstrained biomolecular environment.

TABLE 9

| | | | |
|---|---|---|---|
| δ | Cl | Br | I |

XB Donor: Halobenzene

| | | |
|---|---|---|
| −0.75 kcal/mol | −1.54 kcal/mol | −2.61 kcal/mol |

XB Donor: 2-Halophenol

| | | | |
|---|---|---|---|
| 0° (cis-OH) | −1.67 kcal/mol | −2.36 kcal/mol | −3.37 kcal/mol |
| 90° | −0.76 kcal/mol | −1.55 kcal/mol | −2.64 kcal/mol |
| 180° (trans-OH) | −0.52 kcal/mol | −1.30 kcal/mol | −2.43 kcal/mol |
| Δδ (cis-trans) | −1.15 kcal/mol | −1.06 kcal/mol | −0.94 kcal/mol |

Table 9 shows the quantum mechanical energies for the neutral oxygen halogen bond (XB) acceptor of N-methylacetamide interacting with a halobenzene or 2-halophenol XB donor. As shown, the halogen to oxygen distances (r_{X ⋅ ⋅ ⋅ O}) for both the halobenzene and 2-halophenol XB donors were set to 92% of the sum of the respective van der Waals radii of the halogen and the XB acceptor (r_{Cl ⋅ ⋅ ⋅ O}=3.01 Å, r_{Br ⋅ ⋅ ⋅ O}=3.10 Å, and r_{I ⋅ ⋅ ⋅ O}=3.22 Å). The angle of approach of the acceptor oxygen to the X—C bond (θ₁-angle) was set at optimum value of 180° for all calculations. For the 2-halophenol donor, the energies were calculated with the OH-substituent rotated to align the hydrogen towards the halogen (δ=0°), away from the halogen (δ=180°), or perpendicular to X ⋅ ⋅ ⋅ O (δ=90°). The differences in energies between δ values of 0° and 180° are fairly independent of r_{X ⋅ ⋅ ⋅ O}.

TABLE 10

| | | | |
|---|---|---|---|
| δ | Cl | Br | I |

XB Donor: Halobenzene

| | | |
|---|---|---|
| +0.03 kcal/mol | −1.66 kcal/mol | −4.29 kcal/mol |

XB Donor: 2-Halophenol

| | | | |
|---|---|---|---|
| 0° (cis-OH) | −2.46 kcal/mol | −4.16 kcal/mol | −6.67 kcal/mol |
| 90° | 0.00 kcal/mol | −1.72 kcal/mol | −4.35 kcal/mol |
| 180° (trans-OH) | +1.05 kcal/mol | −0.72 kcal/mol | −3.45 kcal/mol |
| Δδ (cis-trans) | −3.51 kcal/mol | −3.44 kcal/mol | −3.22 kcal/mol |

Table 10 shows the quantum mechanical energies for the anionic oxygen halogen bond (XB) acceptor of hypophosphite interacting with a halobenzene or 2-halophenol XB donor. Here, the halogen to oxygen distances (r_{X ⋅ ⋅ ⋅ O}) for both the halobenzene and 2-halophenol donors were set to those seen in the crystal structures of the Cl2J (r_{X ⋅ ⋅ ⋅ O}=2.88 Å), Br2J (r_{X ⋅ ⋅ ⋅ O}=2.87 Å), and I2J (r_{X ⋅ ⋅ ⋅ O}=3.01 Å) DNA constructs. The angle of approach of the acceptor oxygen to the X—C bond (θ₁-angle) was set at optimum value of 180° for all calculations. For the 2-halophenol donor, the energies were calculated with the OH-substituent rotated to align the hydrogen towards the halogen (δ=0°), away from the halogen (δ=180°), or perpendicular to X ⋅ ⋅ ⋅ O (δ=90°).

### Introduction of Example 6

Protein function often requires the folded protein form, but this form is usually unstable mainly because it readily unfolds into a flexible, unstructured form. An understanding for how to stabilize such proteins could help prevent the formation of protein aggregates, including those (e.g., β-amyloids) that are associated with neurodegenerative diseases. Further, detailed knowledge of the structure and function of a protein may greatly expand the abilities of protein engineering of more stable proteins for commercial use. Proteins engineered to have increased stability can advantageously (1) be used as biocatalysts in artificial environments, (2) have extended lifetimes or storage at ordinary temperatures, (3) result in higher yields of soluble protein during manufacture, and/or (4) be more ‘evolvable’ or able to acquire beneficial traits for a given environment. Thus far, engineered mutations rarely result in significant stabilization (≥1° increase in the melting temperature, T_{M}) of any engineered protein.

In the following study, the metastable KIX domain (kinase-inducible domain) domain of the yeast transcriptional coactivator CBP (also known as CREB-binding protein or CREBBP) was used as the experimental system. The KIX domain is important in mediating protein-protein interactions and is the target for recognition by several other transcription activators, including CREB and c-myc, with the folding state of KIX domain defining how it interacts with various recognition partners.

### Example 6

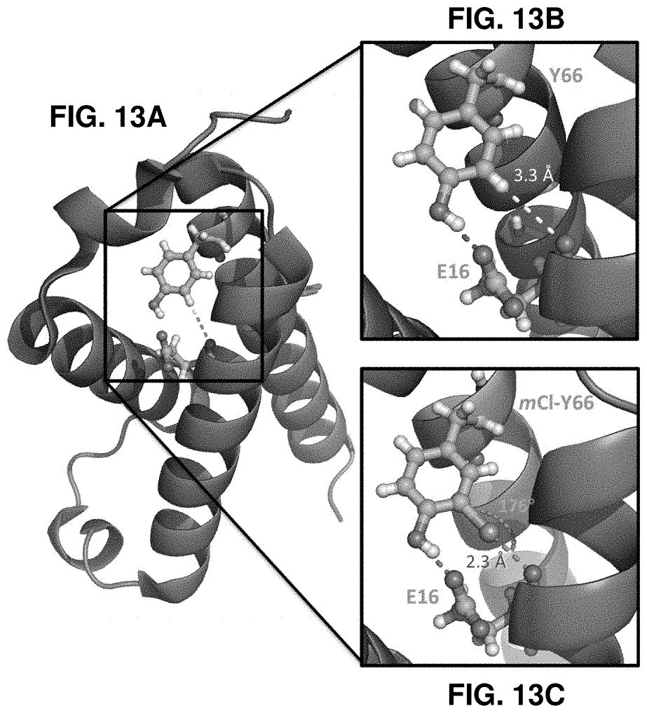

The three helix bundle structure of KIX has been shown by NMR studies to be only 50%-80% folded at 20° C. The tyrosine residue at position 66 (Y66) forms a stabilizing hydrogen bond to the carboxylate side chain of glutamate 16 (E16) of wild type KIX (FIGS. 13A-13B). Tyrosine Y66 was replaced by a meta-halogenated tyrosine forming a chlorinated ^{Cl}Y66 construct (FIG. 13C) or an iodinated ^{I}Y66 construct.

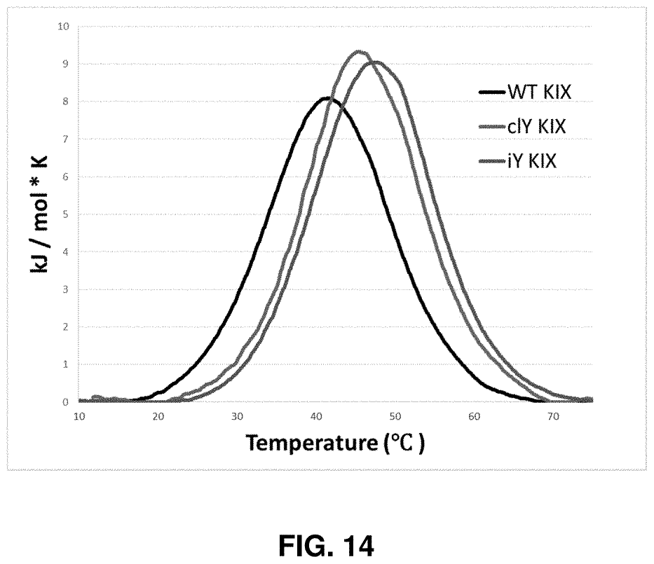

Using differential scanning calorimetry (DSC), the melting temperature (T_{M}, the temperature at which the protein is 50% folded and 50% unfolded) was measured for WT-KIX, the chlorinated ^{Cl}Y66 construct, and the iodinated ^{I}Y66 construct (FIG. 14). T_{M} for WT-KIX was found to be ˜42° C., confirming that the protein is partially folded at ambient temperatures. When Y66 was replaced by a meta-halogenated tyrosine, the T_{M} increases from +4° C. (for ^{Cl}Y66) to +5.7° C. (for ^{I}Y66), reflecting a significant increase in the thermal stability of the protein and increase in the proportion of folded protein at ambient temperatures. The amount of heat required to melt the protein, as measured by the enthalpy of melting (ΔH_{M}) also increased significantly by 20 or 25 kJ/mol (equivalent to ˜5 to 6 kcal/mol), indicating the formation of a strong halogen bond (Table 11).

TABLE 11

| | | | | ΔH_{M} | ΔΔH_{M} |
|---|---|---|---|---|---|
| Construct | Halogen | T_{M} (° C.) | ΔT_{M} (° C.) | (kJ/mol) | (kJ/mol) |
| WT KIX | None | 41.9 ± 0.1 | 0 | 152 ± 3 | 0 |
| ^{Cl}Y KIX | Chlorine | 45.9 ± 0.2 | +4.0 ± 0.2 | 177 ± 4 | 25 ± 5 |
| ^{I}Y KIX | Iodine | 47.6 ± 0.1 | +5.7 ± 0.1 | 172 ± 3 | 20 ± 5 |

A model of the potential interaction, starting with the crystal structure of the homologous human KIX protein, showed a near ideal halogen bond interaction to the carbonyl oxygen of the peptide backbone at E16, with distances less than the sum of the van der Waals radii and linear alignment (C—X ⋅ ⋅ ⋅ O angle of ˜176°). Collectively, data showed that a partially-stable protein can be stabilized by an engineered HeX-bond.

### Methods Used in Examples 1-6

### (a) Site-Directed Mutagenesis and Protein Expression

All T4 lysozyme (T4L) constructs started with the gene of pseudo-wild-type (WT*) protein, the T4L double mutant C54T/C97A, with the DNA sequence encoding a six-His tag appended at the C-terminus to facilitate protein purification. The ^{mX}Y18-T4L constructs (^{mCl}Y18, ^{mBr}Y18, and ^{mI}Y18) had the codon for Y18 replaced with an AMBER (TAG) codon. The modified DNA sequences were inserted into the pBAD vector for DNA amplification in DH5a Escherichia coli.

The expression vector for the WT* construct was transformed into BL21 (DE3) pLysS E. coli. Transformed cells were grown in 2×YT medium with the appropriate antibiotics (ampicillin and chloramphenicol) and incubated at 37° C. while being shaken at 250 rpm until an OD₆₀₀ of 0.4-0.6 was reached. The cells were induced with the addition of arabinose directly to the cultures to a final concentration of 0.2% (w/v) and allowed to grow for an additional 3 hours. Subsequently, the cells were harvested by centrifugation at 2200 relative centrifugal force (RCF). Thereafter, the supernatant was decanted, and the bacterial pellet was stored at −80° C.

Expression vectors for the ^{mX}Y18-T4L constructs were co-transformed into BL21ai E. coli with the pDule2-Mb-CITyrRS-C6 plasmid that contains the orthogonal Mb tRNA_{CUA} and 3-halo-Tyr amino acyl-tRNA synthetase. After being rescued, the transformed cells were stored at −80° C. Starter cultures of NIM medium containing appropriate antibiotics (ampicillin and spectinomycin) were inoculated with these cell stocks and allowed to grow at 37° C. for 12 hours while being shaken at 250 rpm. Then, 5 mL of the starter cultures was used to, in a 2 L culture flask, inoculate 500 mL of AIM medium containing the appropriate antibiotics (ampicillin and spectinomycin), but lacking arabinose. After inoculation, the cultures grew at 37° C. while being shaken at 250 rpm. When an OD₆₀₀ of ˜1.0 was reached, the noncanonical amino acid (3-chloro-I-tyrosine, 3-bromo-I-tyrosine, or 3-iodo-I-tyrosine) was added to the cultures to obtain a final concentration of 1.0 mM. The 3-halo-I-tyrosines were supplied from Ark Pharm, Inc. The cultures continued to grow at 37° C. while being shaken at 250 rpm. When an OD₆₀₀ of 3.0-4.0 was reached, the cultures were induced with a final concentration of 0.2% (w/v) arabinose. After induction, the bacterial growth was continued for 3 hours at 37° C. while the sample was being shaken at a reduced speed of 100 rpm. Finally, after expression for 3 hours, the cells were harvested by centrifugation at 4000 RCF, the supernatants were decanted, and the bacterial pellets were stored at −80° C.

A similar protocol was followed for meta-halogenated tyrosine KIX constructs.

### (b) Protein Purification

The frozen bacterial pellets were suspended in 35-45 mL of a 9:1 buffer A/buffer B mixture [buffer A consisting of 40 mM potassium phosphate (pH 7.4), 500 mM sodium chloride, and 0.02% (w/v) sodium azide and buffer B consisting of 40 mM potassium phosphate (pH 7.4), 500 mM sodium chloride, 500 mM imidazole, and 0.02% (w/v) sodium azide] and thawed in a 37° C. water bath for 15 minutes. Subsequently, the cells were lysed by sonication on ice for 3×30 seconds using a Branson Sonifier 450 sonicator (duty cycle of 70%, output control of 7). After cell lysis, the homogeneous suspension was centrifuged in a Beckman model J2-21 centrifuge equipped with a JA-20 rotor at 16000 rpm and 4° C. for 30 minutes. The supernatant was decanted and filtered twice, first through a 0.45 pm pore syringe filter and thereafter through a 0.22 pm pore filter. The filtered cell lysate was loaded, applying 10% buffer B, onto a 5 mL HisTrap HP column on an AKTA start FPLC system. Nonbound protein was washed out with 15% buffer B over 5 column volumes. The His-tagged T4L construct was eluted with a gradient of 20 to 100% buffer B over 13 column volumes. Selected fractions were combined and concentrated to 1 mL in an Amicon Ultra-15 10K (Millipore) centrifugal device [10000 molecular weight cutoff (MWCO)] in an Eppendorf 5810 R centrifuge at 4000 rpm and 4° C. The concentrated protein solution was then loaded onto a gravity-fed Sephadex G-50 fine column equilibrated in buffer specific for either crystallization or differential scanning calorimetry (DSC) [crystallization buffer consisting of 100 mM sodium phosphate (pH 7.0), 500 sodium chloride, and 0.02% (w/v) sodium azide and DSC buffer consisting of 20 mM glycine-HCl (pH 3.5), 80 mM sodium chloride, and 1 mM EDTA]. After gel filtration, the selected fractions were combined and used for crystallization or DSC experiments.

A similar protocol was followed for wild type (WT) KIX and all meta-halogenated tyrosine KIX constructs.

### (c) Protein Crystallization

After gel filtration purification using the crystallization buffer, described above, the combined and selected fractions were concentrated to 13-20 mg/mL using an Amicon Ultra-15 10K (Millipore) centrifugal device (10000 MWCO) in an Eppendorf 5810 R centrifuge at 4000 rpm and 4° C. Crystals of the ^{mX}Y18-T4L constructs were grown at 18° C. using the hanging drop vapor diffusion method with a 2:3 to 7:3 ratio of protein to precipitant solution [precipitant solutions consisting of 2.0-2.4 M potassium phosphate (pH 6.5-7.4), 50 mM 2-hydroxyethyldisulfide, and 50 mM 2-mercaptoethanol] with a final protein concentration of 8-10 mg/mL in a 3.5-4.0 μL total drop volume, similar to the process previously described. Diffraction quality crystals grew after 2-5 days for the ^{mBr}Y18 and ^{mCl}Y18-T4L constructs and after ˜2 weeks for the ^{mI}Y18-T4L construct. The crystals were harvested using a cryo-loop, flash-frozen, and stored in liquid nitrogen until X-ray data were collected.

### (d) X-Ray Data Collection and Structure Determination

X-ray diffraction data were collected on crystals held under a cryogenic nitrogen stream (100 K) on the Advanced Light Source (ALS) Beamline 4.2.2 at Berkeley National Laboratory (1.00 Å, Research Detectors Inc. complementary metal-oxide-semiconductor 8 M detector). Diffraction data from the ALS beamline were reduced using XDS and the CCP4 suite. X-ray data were phased by molecular replacement, applying the atomic coordinates of WT* [Protein Data Bank (PDB) entry 1L63] as the starting model, yielding initial models with R_{work} values that ranged from 29.6 to 39.5% and R_{free} values that ranged from 30.9 to 41.2%. Subsequent refinement of the structure using the PHENIX suite of crystallographic software resulted in final structures with R_{work} values that ranged from 17.7 to 19.9% and R_{free} values that ranged from 20.8 to 23.2% (Table 1).

### (e) Differential Scanning Calorimetry (DSC)

After gel filtration purification [DSC buffer (pH 3.5)], as described above, the combined fractions of the pure T4L construct were diluted to a concentration of 0.3 mg/mL with DSC buffer. Aliquots of 900 μL were prepared and stored at −80° C. A low pH was used to help promote reversible folding. Melting curves were collected on a TA Instruments Nano DSC model 602001 instrument under constant pressure (3.0 atm) with all samples matched against identical buffer in the reference cell. Samples were equilibrated for 600 seconds, followed by melting data collection through heating cycles from 10° C. to 90° C. at a scan rate of 0.75° C./minute. The reversibility was confirmed for all constructs by performing a cooling scan from 90° C. to 10° C. at a scan rate of 0.5° C./minute and a subsequent heating cycle. A minimum of 10 replicate experiments were conducted for each T4L construct. Melting data were analyzed, and thermodynamic parameters, including the specific heat capacities (ΔCₚ), were determined using NanoAnalyze Data Analysis, version 3.6.0, from TA Instruments. The melting temperatures (T_{M}) and enthalpies (ΔH_{M}) were extracted using the TwoStateScaled model for fitting the experimental data. The ΔH_{fit}/ΔH_{cal} ratios were all in the range of 0.97-1.01; the Aw values were in the range of 0.99-1.05, and the standard deviation of the fits was <1.6 for all experiments.

A similar protocol was followed for wild type (WT) KIX and all meta-halogenated tyrosine KIX constructs.

### (f) Quantum Mechanical (QM) Calculations

QM energies and electrostatic potential maps (ESPs) were calculated using Gaussian 09 revision E.01 with the Møller-Plesset second order (MP2) in a cyclohexane solvent (D=2 relative to a vacuum). This low-dielectric solvent model is appropriate for calculations on systems that involve explicit solvent and short distances between interacting atoms, as is the case in this study, and reflects the low dielectric expected for a protein interior. Basis set superposition errors (BSSEs) were determined from a separate counterpoise gas phase calculation and directly summed into the calculated solvent phase energy. Polarizable basis sets, including dispersion, were applied (aug-cc-PVTZ for ^{mCl}Y18 and ^{mBr}Y18 and extended to ^{mI}Y18 with aug-cc-PVTZ-PP from EMSL basis set exchange). The strategy for QM calculations applied here had previously been validated against experimental XB geometries and energies in model DNA junction systems. The atomic coordinates of the interacting residues (Y18 and G28) were taken from the refined structures of each construct. Residue 18 was reduced to 2-halophenol, and residue 28 was reduced to N-methylacetamide (NMA) to decrease computational time. Hydrogen atom positions were geometry-optimized with a semiempirical AM1 calculation. The torsional angle, δ, of the hydroxyl hydrogen is manually rotated to determine its contribution to the HeX-B.

### (g) Turbidity Assay

The activities of the T4L constructs were monitored through a standard cell clearing assay. Microccocus lysodeikitcus bacteria were grown in 50 mL of 2×YT medium overnight at 37° C. while being shaken at 250 rpm. Then, the culture was centrifuged in an Eppendorf 5810 R centrifuge at 4000 rpm at 4° C. for 15 minutes. The supernatant was decanted, and the cell pellet diluted in a 1:1 mixture of 50 mM monobasic and 50 mM dibasic sodium phosphate solutions until an OD₄₅₀ of 1.0 was reached. Bacterial samples of 1.0 mL were prepared and stored at −80° C. After the samples had thawed, the purified and concentrated T4L construct in crystallization buffer was added to the bacterial sample to reach a final concentration of 0.1 mg/mL. The absorbance change over time was measured at room temperature (23° C.) and 40° C. Three or four replicates of each construct were run for each temperature.

1. A method of increasing the stability of an engineered protein, the method comprising formation of a hydrogen bond-enhanced halogen bond (HeX-B) by halogenating at least one amino acid residue of the protein wherein the thermal stability of the engineered protein is higher than a parent protein under the same conditions.

2. The method of claim 1, wherein the halogen atom is selected from fluorine, chlorine, bromine, or iodine.

3. The method of claim 1, wherein the halogen atom is added to the at least one amino acid residue at the meta-position.

4. The method of claim 1, wherein the halogen bond (XB) forms an electropositive σ-hole, and wherein the XB further forms an electronegative annulus around the center of the bond.

5. (canceled)

6. The method of claim 4, wherein the hydrogen bond (HB) acts as an electron donor, and wherein the HB intensifies the electropositive σ-hole.

7. (canceled)

8. (canceled)

9. The method of claim 1, wherein the engineered protein has a melting temperature that is at least 0.5° C. higher than the parent protein.

10. (canceled)

11. The method of claim 1, wherein the engineered protein has an enthalpy (ΔH_{M}) that is at least 1 kcal/mol higher than the parent protein.

12. (canceled)

13. The method of claim 1, wherein the engineered protein is an engineered enzyme, a structural protein, a signaling protein, a regulatory protein, a transport protein, a sensory protein, a motor protein, a defense protein, a hormonal protein, or a storage protein.

14. The method of claim 1, wherein the engineered protein is an engineered enzyme, and the enzymatic activity of the engineered enzyme is higher than a parent enzyme under the same conditions.

15. (canceled)

16. The method of claim 1, wherein the halogen on the at least one amino acid residue is at least partially unexposed to solvent.

17. (canceled)

18. An engineered protein comprising at least one halogenated amino acid residue, wherein the halogenated amino acid residue comprises formation of a hydrogen bond-enhanced halogen bond (HeX-B) which thermally stabilizes the engineered protein as compared to a parent protein.

19. The engineered protein of claim 18, wherein the halogenated amino acid residue comprises a halogen atom selected from fluorine, chlorine, bromine, or iodine.

20. The engineered protein of claim 18, wherein the halogen atom is added to the amino acid residue at the meta-position.

21. The engineered protein of claim 18, wherein the XB forms an electropositive σ-hole, and wherein the XB further forms an electronegative annulus around the center of the bond.

22. (canceled)

23. The engineered protein of claim 21, wherein the HB acts as an electron donor, and wherein the HB intensifies the electropositive σ-hole.

24. (canceled)

25. (canceled)

26. The engineered protein of claim 18, wherein the engineered protein is an engineered enzyme, a structural protein, a signaling protein, a regulatory protein, a transport protein, a sensory protein, a motor protein, a defense protein, a hormonal protein, or a storage protein.

27. The engineered protein of claim 18, wherein the engineered protein is an engineered enzyme, wherein enzymatic activity of the engineered enzyme is higher than a parent enzyme under the same conditions.

28. (canceled)

29. The engineered protein of claim 18, wherein the halogen on the at least one amino acid residue is at least partially unexposed to solvent.

30. (canceled)

31. The engineered protein of claim 18, wherein the engineered protein has a melting temperature that is at least 0.5° C. higher than the parent protein.

32. (canceled)

33. The engineered protein of claim 18, wherein the engineered protein has an enthalpy (ΔH_{M}) that is at least 1 kcal/mol higher than the parent protein.

34. (canceled)
